SEQUENCE LISTING (97 sequences)

SEQ ID NO: 1 - protein, 15 aa, synthetic construct
  REGION 1..15: note = synthetic
CSWSWLTMCQKLHLC

SEQ ID NO: 2 - protein, 15 aa, synthetic construct
  REGION 1..15: note = synthetic
CSTSWMNLCKQLNLC

SEQ ID NO: 3 - protein, 15 aa, synthetic construct
  REGION 1..15: note = synthetic
CSPTWTNTCIQLGLC

SEQ ID NO: 4 - protein, 15 aa, synthetic construct
  REGION 1..15: note = synthetic
CTPNWNAMCLKLNLC

SEQ ID NO: 5 - protein, 14 aa, synthetic construct
  REGION 1..14: note = synthetic
CDVFTHCILLAKPC

SEQ ID NO: 6 - protein, 15 aa, synthetic construct
  REGION 1..15: note = synthetic
CSESWSNMCVSLGLC

SEQ ID NO: 7 - protein, 15 aa, synthetic construct
  REGION 1..15: note = synthetic
CSAEWRNMCVQLDLC

SEQ ID NO: 8 - protein, 15 aa, synthetic construct
  REGION 1..15: note = synthetic
CSASWSNMCVELGLC

SEQ ID NO: 9 - protein, 15 aa, synthetic construct
  REGION 1..15: note = synthetic
CSDNWLNMCVELGLC

SEQ ID NO: 10 - protein, 15 aa, synthetic construct
  REGION 1..15: note = synthetic
CSESWSAMCASLGLC

SEQ ID NO: 11 - protein, 15 aa, synthetic construct
  REGION 1..15: note = synthetic
CSESWSNMCKSLGLC

SEQ ID NO: 12 - protein, 15 aa, synthetic construct
  REGION 1..15: note = synthetic
CSESWRNMCVQLNLC

SEQ ID NO: 13 - protein, 15 aa, synthetic construct
  REGION 1..15: note = synthetic
CSPEWTNMCVQLHLC

SEQ ID NO: 14 - protein, 15 aa, synthetic construct
  REGION 1..15: note = synthetic
CSTQWNNMCVQLGLC

SEQ ID NO: 15 - protein, 15 aa, synthetic construct
  REGION 1..15: note = synthetic
CSSSWTNMCVQLGLC

SEQ ID NO: 16 - protein, 15 aa, synthetic construct
  REGION 1..15: note = synthetic
CSAEWRNMCVELNLC

SEQ ID NO: 17 - protein, 15 aa, synthetic construct
  REGION 1..15: note = synthetic
CSPEWKNMCITLNLC

SEQ ID NO: 18 - protein, 15 aa, synthetic construct
  REGION 1..15: note = synthetic
  MOD_RES 2: note = X represents homoarginine
CXDWCHWTFSHGHPC

SEQ ID NO: 19 - protein, 15 aa, synthetic construct
  REGION 1..15: note = synthetic
CSAGWLTMCQKLHLC

SEQ ID NO: 20 - protein, 15 aa, synthetic construct
  REGION 1..15: note = synthetic
  MOD_RES 11: note = X represents lysine-4-pentynoic acid
CSAGWLTMCQXLHLC

SEQ ID NO: 21 - protein, 15 aa, synthetic construct
  REGION 1..15: note = synthetic
  MOD_RES 3: note = X represents Aib
CSXGWLTMCQKLHLC

SEQ ID NO: 22 - protein, 15 aa, synthetic construct
  REGION 1..15: note = synthetic
  MOD_RES 3: note = X represents Abu
CSXGWLTMCQKLHLC

SEQ ID NO: 23 - protein, 15 aa, synthetic construct
  REGION 1..15: note = synthetic
  MOD_RES 4: note = X represents homoserine
CSAXWLTMCQKLHLC

SEQ ID NO: 24 - protein, 15 aa, synthetic construct
  REGION 1..15: note = synthetic
  MOD_RES 8: note = X represents norleucine
CSAGWLTXCQKLHLC

SEQ ID NO: 25 - protein, 15 aa, synthetic construct
  REGION 1..15: note = synthetic
  SITE 3: note = X represents D-Alanine
CSXGWLTMCQKLHLC

SEQ ID NO: 26 - protein, 15 aa, synthetic construct
  REGION 1..15: note = synthetic
  MOD_RES 3: note = X represents homoserine
CSXGWLTMCQKLHLC

SEQ ID NO: 27 - protein, 15 aa, synthetic construct
  REGION 1..15: note = synthetic
  MOD_RES 3: note = X represents norleucine
CSXGWLTMCQKLHLC

SEQ ID NO: 28 - protein, 15 aa, synthetic construct
  REGION 1..15: note = synthetic
  MOD_RES 8: note = X represents homophenylalanine
CSAGWLTXCQKLHLC

SEQ ID NO: 29 - protein, 15 aa, synthetic construct
  REGION 1..15: note = synthetic
CSAGWLTMCVQLGLC

SEQ ID NO: 30 - protein, 15 aa, synthetic construct
  REGION 1..15: note = synthetic
  MOD_RES 10: note = X represents Cyclohexylglycine
CSAGWLTMCXKLHLC

SEQ ID NO: 31 - protein, 15 aa, synthetic construct
  REGION 1..15: note = synthetic
CSAGWLTMCLKLHLC

SEQ ID NO: 32 - protein, 15 aa, synthetic construct
  REGION 1..15: note = synthetic
  MOD_RES 11: note = X represents norleucine
CSAGWLTMCQXLHLC

SEQ ID NO: 33 - protein, 15 aa, synthetic construct
  REGION 1..15: note = synthetic
  MOD_RES 11: note = X represents homoserine
CSAGWLTMCQXLHLC

SEQ ID NO: 34 - protein, 15 aa, synthetic construct
  REGION 1..15: note = synthetic
  MOD_RES 12: note = X represents norleucine
CSAGWLTMCQKXHLC

SEQ ID NO: 35 - protein, 15 aa, synthetic construct
  REGION 1..15: note = synthetic
  SITE 13: note = X represents D-Alanine
CSAGWLTMCQKLXLC

SEQ ID NO: 36 - protein, 15 aa, synthetic construct
  REGION 1..15: note = synthetic
  MOD_RES 13: note = X represents Aib
CSAGWLTMCQKLXLC

SEQ ID NO: 37 - protein, 15 aa, synthetic construct
  REGION 1..15: note = synthetic
  MOD_RES 14: note = X represents norleucine
CSAGWLTMCQKLHXC

SEQ ID NO: 38 - protein, 15 aa, synthetic construct
  REGION 1..15: note = synthetic
CSAGWLTMCQRLHLC

SEQ ID NO: 39 - protein, 15 aa, synthetic construct
  REGION 1..15: note = synthetic
  MOD_RES 14: note = X represents Nva
CSAGWLTMCQKLHXC

SEQ ID NO: 40 - protein, 15 aa, synthetic construct
  REGION 1..15: note = synthetic
  MOD_RES 12: note = X represents Nva
CSAGWLTMCQKXHLC

SEQ ID NO: 41 - protein, 15 aa, synthetic construct
  REGION 1..15: note = synthetic
  MOD_RES 8: note = X represents Nva
CSAGWLTXCQKLHLC

SEQ ID NO: 42 - protein, 15 aa, synthetic construct
  REGION 1..15: note = synthetic
CSAGWLTMCKQLNLC

SEQ ID NO: 43 - protein, 15 aa, synthetic construct
  REGION 1..15: note = synthetic
  MOD_RES 3: note = X represents Aib
  MOD_RES 8: note = X represents homophenylalanine
  MOD_RES 13: note = X represents Aib
CSXGWLTXCLKLXLC

SEQ ID NO: 44 - protein, 15 aa, synthetic construct
  REGION 1..15: note = synthetic
  MOD_RES 5: note = X represents 7-azatryptophan
CSAGXLTMCQKLHLC

SEQ ID NO: 45 - protein, 15 aa, synthetic construct
  REGION 1..15: note = synthetic
  MOD_RES 10: note = X represents 2-aminoadipic acid
CSAGWLTMCXKLHLC

SEQ ID NO: 46 - protein, 15 aa, synthetic construct
  REGION 1..15: note = synthetic
CSAEWLTMCQKLHLC

SEQ ID NO: 47 - protein, 15 aa, synthetic construct
  REGION 1..15: note = synthetic
  MOD_RES 4: note = X represents Aad
CSAXWLTMCQKLHLC

SEQ ID NO: 48 - protein, 15 aa, synthetic construct
  REGION 1..15: note = synthetic
CSAGWLTMCEKLHLC

SEQ ID NO: 49 - protein, 15 aa, synthetic construct
  REGION 1..15: note = synthetic
  MOD_RES 10: note = X represents beta-cyclopropylalanine
CSAGWLTMCXKLHLC

SEQ ID NO: 50 - protein, 15 aa, synthetic construct
  REGION 1..15: note = synthetic
  MOD_RES 2: note = X represents allothreonine
CXAGWLTMCQKLHLC

SEQ ID NO: 51 - protein, 15 aa, synthetic construct
  REGION 1..15: note = synthetic
  MOD_RES 6: note = X represents beta-cyclopropylalanine
CSAGWXTMCQKLHLC

SEQ ID NO: 52 - protein, 15 aa, synthetic construct
  REGION 1..15: note = synthetic
  MOD_RES 14: note = X represents beta-cyclopropylalanine
CSAGWLTMCQKLHXC

SEQ ID NO: 53 - protein, 15 aa, synthetic construct
  REGION 1..15: note = synthetic
  MOD_RES 3: note = X represents norleucine
  MOD_RES 8: note = X represents homophenylalanine
  MOD_RES 13: note = X represents Aib
CSXGWLTXCLKLXLC

SEQ ID NO: 54 - protein, 15 aa, synthetic construct
  REGION 1..15: note = synthetic
CSEGWLTMCQKLHLC

SEQ ID NO: 55 - protein, 15 aa, synthetic construct
  REGION 1..15: note = synthetic
  MOD_RES 3: note = X represents Aad
CSXGWLTMCQKLHLC

SEQ ID NO: 56 - protein, 14 aa, synthetic construct
  REGION 1..14: note = synthetic
CSDQWMQMCSKLTC

SEQ ID NO: 57 - protein, 15 aa, synthetic construct
  REGION 1..15: note = synthetic
CSDGWLTMCQKLHLC

SEQ ID NO: 58 - protein, 15 aa, synthetic construct
  REGION 1..15: note = synthetic
CSDEWLTMCQKLHLC

SEQ ID NO: 59 - protein, 15 aa, synthetic construct
  REGION 1..15: note = synthetic
CSNSWLTMCQKLHLC

SEQ ID NO: 60 - protein, 15 aa, synthetic construct
  REGION 1..15: note = synthetic
CSPAWLTMCQKLHLC

SEQ ID NO: 61 - protein, 15 aa, synthetic construct
  REGION 1..15: note = synthetic
CSPEWLTMCQKLHLC

SEQ ID NO: 62 - protein, 15 aa, synthetic construct
  REGION 1..15: note = synthetic
CSPGWLTMCQKLHLC

SEQ ID NO: 63 - protein, 15 aa, synthetic construct
  REGION 1..15: note = synthetic
CSPQWLTMCQKLHLC

SEQ ID NO: 64 - protein, 15 aa, synthetic construct
  REGION 1..15: note = synthetic
CSPSWLTMCQKLHLC

SEQ ID NO: 65 - protein, 15 aa, synthetic construct
  REGION 1..15: note = synthetic
CSDSWKTMCQKLHLC

SEQ ID NO: 66 - protein, 15 aa, synthetic construct
  REGION 1..15: note = synthetic
CSESWSTMCQKLHLC

SEQ ID NO: 67 - protein, 15 aa, synthetic construct
  REGION 1..15: note = synthetic
CSPSWRTMCQKLHLC

SEQ ID NO: 68 - protein, 15 aa, synthetic construct
  REGION 1..15: note = synthetic
CSPSWRNMCQKLHLC

SEQ ID NO: 69 - protein, 15 aa, synthetic construct
  REGION 1..15: note = synthetic
CSWSWLTMCKQLNLC

SEQ ID NO: 70 - protein, 15 aa, synthetic construct
  REGION 1..15: note = synthetic
CSWSWLTMCQKLDLC

SEQ ID NO: 71 - protein, 15 aa, synthetic construct
  REGION 1..15: note = synthetic
CSSSWLTMCQKLHLC

SEQ ID NO: 72 - protein, 15 aa, synthetic construct
  REGION 1..15: note = synthetic
CSWSWLNMCQKLHLC

SEQ ID NO: 73 - protein, 15 aa, synthetic construct
  REGION 1..15: note = synthetic
CDPLCLSIRASLGLC

SEQ ID NO: 74 - protein, 15 aa, synthetic construct
  REGION 1..15: note = synthetic
CDPLCLSIKRSLGLC

SEQ ID NO: 75 - protein, 15 aa, synthetic construct
  REGION 1..15: note = synthetic
CDPLCLSIKRQLGLC

SEQ ID NO: 76 - protein, 15 aa, synthetic construct
  REGION 1..15: note = synthetic
CDPLCLSIKRKLGLC

SEQ ID NO: 77 - protein, 15 aa, synthetic construct
  REGION 1..15: note = synthetic
CDPLCLSIKRGLGLC

SEQ ID NO: 78 - protein, 15 aa, synthetic construct
  REGION 1..15: note = synthetic
CDMRCIRIKQSLGMC

SEQ ID NO: 79 - protein, 15 aa, synthetic construct
  REGION 1..15: note = synthetic
CRDWCHWTFDNGHPC

SEQ ID NO: 80 - protein, 15 aa, synthetic construct
  REGION 1..15: note = synthetic
CRDWCHWTFSHGTPC

SEQ ID NO: 81 - protein, 15 aa, synthetic construct
  REGION 1..15: note = synthetic
CRDWCHWTFSHGHPC

SEQ ID NO: 82 - protein, 15 aa, synthetic construct
  REGION 1..15: note = synthetic
CRDWCHWTFTHGHPC

SEQ ID NO: 83 - protein, 15 aa, synthetic construct
  REGION 1..15: note = synthetic
CRDWCHWTFTHSHPC

SEQ ID NO: 84 - protein, 15 aa, synthetic construct
  REGION 1..15: note = synthetic
  MOD_RES 3: note = X represents Aad
CSXGWLTMCQKLHLC

SEQ ID NO: 85 - protein, 15 aa, synthetic construct
  REGION 1..15: note = synthetic
  MOD_RES 3: note = X represents Aad
CSXGWLTMCLKLHLC

SEQ ID NO: 86 - protein, 15 aa, synthetic construct
  REGION 1..15: note = synthetic
  MOD_RES 3: note = X represents Aad
  MOD_RES 7: note = X represents 2-amino-3-hydroxy-3-methyl-butyric acid
CSXGWLXMCLKLHLC

SEQ ID NO: 87 - protein, 15 aa, synthetic construct
  REGION 1..15: note = synthetic
  MOD_RES 11: note = X represents acetyllysine
CSKGWLTMCQXLHLC

SEQ ID NO: 88 - protein, 15 aa, synthetic construct
  REGION 1..15: note = synthetic
  MOD_RES 11: note = X represents acetyllysine
CSAGWLTKCQXLHLC

SEQ ID NO: 89 - protein, 15 aa, synthetic construct
  REGION 1..15: note = synthetic
  MOD_RES 11: note = X represents acetyllysine
CSAGWLTMCKXLHLC

SEQ ID NO: 90 - protein, 15 aa, synthetic construct
  REGION 1..15: note = synthetic
  MOD_RES 11: note = X represents acetyllysine
CSAGWLTMCQXLKLC

SEQ ID NO: 91 - protein, 15 aa, synthetic construct
  REGION 1..15: note = synthetic
  MOD_RES 11: note = X represents homoarginine
CSAGWLTMCQXLHLC

SEQ ID NO: 92 - protein, 15 aa, synthetic construct
  REGION 1..15: note = synthetic
  MOD_RES 10: note = X represents homoarginine
CSAGWLTMCXQLNLC

SEQ ID NO: 93 - protein, 15 aa, synthetic construct
  REGION 1..15: note = synthetic
  MOD_RES 3: note = X represents Aad
CSXGWLTMCKQLNLC

SEQ ID NO: 94 - protein, 15 aa, synthetic construct
  REGION 1..15: note = synthetic
  MOD_RES 3: note = X represents Aad
  MOD_RES 5: note = X represents 3-(1-Naphthyl)-L-alanine
CSXGXLTMCKQLNLC

SEQ ID NO: 95 - protein, 15 aa, synthetic construct
  REGION 1..15: note = synthetic
  MOD_RES 1: note = X represents penicillamine
  MOD_RES 3: note = X represents Aad
XSXGWLTMCKQLNLC

SEQ ID NO: 96 - protein, 15 aa, synthetic construct
  REGION 1..15: note = synthetic
  MOD_RES 3: note = X represents Aad
  MOD_RES 9: note = X represents penicillamine
CSXGWLTMXKQLNLC

SEQ ID NO: 97 - protein, 15 aa, synthetic construct
  REGION 1..15: note = synthetic
  MOD_RES 3: note = X represents Aad
  MOD_RES 15: note = X represents penicillamine
CSXGWLTMCKQLNLX

(19) United States
(12) Patent Application Publication
(10) Pub. No.: US 2026/0152528 A1
(43) Pub. Date: Jun. 4, 2026

(54) BICYCLIC PEPTIDE LIGANDS SPECIFIC FOR PD-L1

(71) Applicant: BicycleTx Limited, Cambridge (GB)

(72) Inventors: Liuhong Chen, Cambridge (GB); James Cooke, Cambridge (GB); Kevin McDonnell, Cambridge (GB); Gemma Elizabeth Mudd, Cambridge (GB); Katerine van Rietschoten, Cambridge (GB); Punit Upadhyaya, Cambridge (GB)

(73) Assignee: BicycleTx Limited, Cambridge (GB)

(21) Appl. No.: 19/380,611

(22) Filed: Nov. 5, 2025

Related U.S. Application Data
(62) Division of application No. 17/044,868, now Pat. No. 12,492,224.
(60) Provisional application No. 62/910,113, filed on Oct. 3, 2019.

(30) Foreign Application Priority Data
Dec. 21, 2018 (GB) 1820956.9
Apr. 2, 2019 (GB) 1904621.8
Apr. 23, 2019 (GB) 1905631.6

(51) Int. Cl. C07K 7/08; A61K 47/64; A61K 49/00; C07K 7/64

(52) U.S. Cl. CPC C07K 7/08; A61K 47/64; A61K 49/0056; C07K 7/64

(57) ABSTRACT

The present invention relates to polypeptides which are covalently bound to non-aromatic molecular scaffolds such that two or more peptide loops are subtended between attachment points to the scaffold. In particular, the invention describes peptides which are high affinity binders of PD-L1. The invention also includes drug conjugates comprising said peptides, conjugated to one or more effector and/or functional groups, to pharmaceutical compositions comprising said peptide ligands and drug conjugates and to the use of said peptide ligands and drug conjugates in preventing, suppressing or treating a disease or disorder mediated by PD-L1.

### RELATED APPLICATIONS

This application is a divisional of U.S. application Ser. No. 17/044,868, filed Oct. 2, 2020, which is a national stage filing under 35 U.S.C. § 371 of international application number PCT/GB2019/053679, filed Dec. 23, 2019, which was published under PCT Article 21(2) in English and claims the benefit under 35 U.S.C. § 119(e) of U.S. provisional application Ser. No. 62/910,113, filed Oct. 3, 2019, United Kingdom application serial number 1905631.6, filed Apr. 23, 2019, United Kingdom application serial number 1904621.8, filed Apr. 2, 2019 and United Kingdom application serial number 1820956.9, filed Dec. 21, 2018, each of which is herein incorporated by reference in its entirety.

### REFERENCE TO AN ELECTRONIC SEQUENCE LISTING

The contents of the electronic sequence listing (B170170046US02-SEQ-HJD.xml; Size: 141,542 bytes; and Date of Creation: Nov. 4, 2025) is herein incorporated by reference in their entirety.

### FIELD OF THE INVENTION

The present invention relates to polypeptides which are covalently bound to non-aromatic molecular scaffolds such that two or more peptide loops are subtended between attachment points to the scaffold. In particular, the invention describes peptides which are high affinity binders of PD-L1. The invention also includes drug conjugates comprising said peptides, conjugated to one or more effector and/or functional groups, to pharmaceutical compositions comprising said peptide ligands and drug conjugates and to the use of said peptide ligands and drug conjugates in preventing, suppressing or treating a disease or disorder mediated by PD-L1.

### BACKGROUND OF THE INVENTION

Cyclic peptides are able to bind with high affinity and target specificity to protein targets and hence are an attractive molecule class for the development of therapeutics. In fact, several cyclic peptides are already successfully used in the clinic, as for example the antibacterial peptide vancomycin, the immunosuppressant drug cyclosporine or the anti-cancer drug octreotide (Driggers et al. (2008), Nat Rev Drug Discov 7(7), 608-24). Good binding properties result from a relatively large interaction surface formed between the peptide and the target as well as the reduced conformational flexibility of the cyclic structures. Typically, macrocycles bind to surfaces of several hundred square angstrom, as for example the cyclic peptide CXCR4 antagonist CVX15 (400 Å²; Wu et al. (2007), Science 330, 1066-71), a cyclic peptide with the Arg-Gly-Asp motif binding to integrin αVb3 (355 Å²) (Xiong et al. (2002), Science 296 (5565), 151-5) or the cyclic peptide inhibitor upain-1 binding to urokinase-type plasminogen activator (603 Å²; Zhao et al. (2007), J Struct Biol 160 (1), 1-10).

Due to their cyclic configuration, peptide macrocycles are less flexible than linear peptides, leading to a smaller loss of entropy upon binding to targets and resulting in a higher binding affinity. The reduced flexibility also leads to locking target-specific conformations, increasing binding specificity compared to linear peptides. This effect has been exemplified by a potent and selective inhibitor of matrix metalloproteinase 8 (MMP-8) which lost its selectivity over other MMPs when its ring was opened (Cherney et al. (1998), J Med Chem 41 (11), 1749-51). The favorable binding properties achieved through macrocyclization are even more pronounced in multicyclic peptides having more than one peptide ring as for example in vancomycin, nisin and actinomycin.

Different research teams have previously tethered polypeptides with cysteine residues to a synthetic molecular structure (Kemp and McNamara (1985), J. Org. Chem; Timmerman et al. (2005), ChemBioChem). Meloen and co-workers had used tris(bromomethyl)benzene and related molecules for rapid and quantitative cyclisation of multiple peptide loops onto synthetic scaffolds for structural mimicry of protein surfaces (Timmerman et al. (2005), ChemBioChem). Methods for the generation of candidate drug compounds wherein said compounds are generated by linking cysteine containing polypeptides to a molecular scaffold as for example 1,1′,1″-(1,3,5-triazinane-1,3,5-triyl)triprop-2-en-1-one (TATA) (Heinis et al(2014) Angewandte Chemie, International Edition 53(6) 1602-1606).

Phage display-based combinatorial approaches have been developed to generate and screen large libraries of bicyclic peptides to targets of interest (Heinis et al. (2009), Nat Chem Biol 5 (7), 502-7 and WO 2009/098450). Briefly, combinatorial libraries of linear peptides containing three cysteine residues and two regions of six random amino acids (Cys-(Xaa)₆-Cys-(Xaa)₆-Cys) were displayed on phage and cyclised by covalently linking the cysteine side chains to a small molecule scaffold.

### SUMMARY OF THE INVENTION

According to a first aspect of the invention, there is provided a peptide ligand specific for PD-L1 comprising a polypeptide comprising at least three reactive groups, separated by at least two loop sequences, and a non-aromatic molecular scaffold which forms covalent bonds with the reactive groups of the polypeptide such that at least two polypeptide loops are formed on the molecular scaffold.

According to a further aspect of the invention, there is provided a drug conjugate comprising a peptide ligand as defined herein conjugated to one or more effector and/or functional groups.

According to a further aspect of the invention, there is provided a pharmaceutical composition comprising a peptide ligand or a drug conjugate as defined herein in combination with one or more pharmaceutically acceptable excipients.

According to a further aspect of the invention, there is provided a peptide ligand or drug conjugate as defined herein for use in preventing, suppressing or treating a disease or disorder mediated by PD-L1.

### BRIEF DESCRIPTION OF THE FIGURES

FIG. 1: PD-1/PD-L1 Blockade Bioassay results with BCY10467, BCY10939 and BCY10959.

### DETAILED DESCRIPTION OF THE INVENTION

According to one particular aspect of the invention which may be mentioned, there is provided a peptide ligand specific for PD-L1 comprising a polypeptide comprising at least three cysteine residues, separated by at least two loop sequences, and a non-aromatic molecular scaffold which forms covalent bonds with the cysteine residues of the polypeptide such that at least two polypeptide loops are formed on the molecular scaffold.

In one embodiment, said loop sequences comprise 3, 5, 6, 7 or 9 amino acids.

In a further embodiment, said loop sequences comprise three reactive groups separated by two loop sequences a first of which consists of 7 amino acids and a second of which consists of 5 amino acids.

In a further embodiment, said loop sequences comprise three reactive groups (i.e. two Cys residues and one Pen residue) separated by two loop sequences a first of which consists of 7 amino acids and a second of which consists of 5 amino acids.

In a further embodiment, said loop sequences comprise three cysteine residues separated by two loop sequences a first of which consists of 7 amino acids and a second of which consists of 5 amino acids.

In a further embodiment, said loop sequences comprise three reactive groups separated by two loop sequences a first of which consists of 5 amino acids and a second of which consists of 6 amino acids.

In a further embodiment, said loop sequences comprise three cysteine residues separated by two loop sequences a first of which consists of 5 amino acids and a second of which consists of 6 amino acids.

In a further embodiment, said loop sequences comprise three reactive groups separated by two loop sequences a first of which consists of 3 amino acids and a second of which consists of 9 amino acids.

In a further embodiment, said loop sequences comprise three cysteine residues separated by two loop sequences a first of which consists of 3 amino acids and a second of which consists of 9 amino acids.

In a further embodiment, said loop sequences comprise three reactive groups separated by two loop sequences a first of which consists of 7 amino acids and a second of which consists of 4 amino acids.

In a further embodiment, said loop sequences comprise three cysteine residues separated by two loop sequences a first of which consists of 7 amino acids and a second of which consists of 4 amino acids.

In one embodiment, said peptide ligand comprises an amino acid sequence selected from:

(SEQ ID NO: 1)
CᵢSWSWLTMCᵢᵢQKLHLCᵢᵢᵢ;
(SEQ ID NO: 2)
CᵢSTSWMNLCᵢᵢKQLNLCᵢᵢᵢ;
(SEQ ID NO: 3)
CᵢSPIWTNTCᵢᵢIQLGLCᵢᵢᵢ;
(SEQ ID NO: 4)
CᵢTPNWNAMCᵢᵢLKLNLCᵢᵢᵢ;
(SEQ ID NO: 5)
CᵢDVFTHCᵢᵢILLAKPCᵢᵢᵢ;
(SEQ ID NO: 6)
CᵢSESWSNMCᵢᵢVSLGLCᵢᵢᵢ;
(SEQ ID NO: 7)
CᵢSAEWRNMCᵢᵢVQLDLCᵢᵢᵢ;
(SEQ ID NO: 8)
CᵢSASWSNMCᵢᵢVELGLCᵢᵢᵢ;
(SEQ ID NO: 9)
CᵢSDNWLNMCᵢᵢVELGLCᵢᵢᵢ;
(SEQ ID NO: 10)
CᵢSESWSAMCᵢᵢASLGLCᵢᵢᵢ;
(SEQ ID NO: 11)
CᵢSESWSNMCᵢᵢKSLGLCᵢᵢᵢ;
(SEQ ID NO: 12)
CᵢSESWRNMCᵢᵢVQLNLCᵢᵢᵢ;
(SEQ ID NO: 13)
CᵢSPEWTNMCᵢᵢVQLHLCᵢᵢᵢ;
(SEQ ID NO: 14)
CᵢSTQWNNMCᵢᵢVQLGLCᵢᵢᵢ;
(SEQ ID NO: 18)
CᵢSSSWTNMCᵢᵢVQLGLCᵢᵢᵢ;
(SEQ ID NO: 16)
CᵢSAEWRNMCᵢᵢVELNLCᵢᵢᵢ;
(SEQ ID NO: 17)
CᵢSPEWKNMCᵢᵢITLNLCᵢᵢᵢ;
(SEQ ID NO: 18)
Cᵢ[HArg]DWCᵢᵢHWTFSHGHPCᵢᵢᵢ;
(SEQ ID NO: 19)
CᵢSAGWLTMCᵢᵢQKLHLCᵢᵢᵢ;
(SEQ ID NO: 20)
CᵢSAGWLTMCᵢᵢQ[K(PYA)]LHLCᵢᵢᵢ;
(SEQ ID NO: 21)
CᵢS[Aib]GWLTMCᵢᵢQKLHLCᵢᵢᵢ;
(SEQ ID NO: 22)
CᵢS[Abu]GWLTMCᵢᵢQKLHLCᵢᵢᵢ;
(SEQ ID NO: 23)
CᵢSA[HSer]NLTMCᵢᵢQKLHLCᵢᵢᵢ;
(SEQ ID NO: 24)
CᵢSAGWLT[Nle]CᵢᵢQKLHLCᵢᵢᵢ;
(SEQ ID NO: 25)
CᵢS[dA]GWLTMCᵢᵢQKLHLCᵢᵢᵢ;
(SEQ ID NO: 26)
CᵢS[HSer]GWLTMCᵢᵢQKLHLCᵢᵢᵢ;
(SEQ ID NO: 27)
CᵢS[Nle]GWLTMCᵢᵢQKLHLCᵢᵢᵢ;
(SEQ ID NO: 28)
CᵢSAGWLT[HPhe]CᵢᵢQKLHLCᵢᵢᵢ;
(SEQ ID NO: 29)
CᵢSAGWLTMCᵢᵢVQLGLCᵢᵢᵢ;
(SEQ ID NO: 30)
CᵢSAGWLTMCᵢᵢ[Chg]KLHLCᵢᵢᵢ;
(SEQ ID NO: 31)
CᵢSAGWLTMCᵢᵢLKLHLCᵢᵢᵢ;
(SEQ ID NO: 32)
CᵢSAGWLTMCᵢᵢQ[Nle]LHLCᵢᵢᵢ;
(SEQ ID NO: 33)
CᵢSAGWLTMCᵢᵢQ[HSer]LHLCᵢᵢᵢ;
(SEQ ID NO: 34)
CᵢSAGWLTMCᵢᵢQK[Nle]HLCᵢᵢᵢ;
(SEQ ID NO: 35)
CᵢSAGWLTMCᵢᵢQKL[dA]LCᵢᵢᵢ;
(SEQ ID NO: 36)
CᵢSAGWLTMCᵢᵢQKL[Aib]LCᵢᵢᵢ;
(SEQ ID NO: 37)
CᵢSAGWLTMCᵢᵢQKLH[Nle]Cᵢᵢᵢ;
(SEQ ID NO: 38)
CᵢSAGWLTMCᵢᵢQRLHLCᵢᵢᵢ;
(SEQ ID NO: 39)
CᵢSAGWLTMCᵢᵢQKLH[Nva]Cᵢᵢᵢ;
(SEQ ID NO: 40)
CᵢSAGWLTMCᵢᵢQK[Nva]HLCᵢᵢᵢ;
(SEQ ID NO: 41)
CᵢSAGWLT[Nva]CᵢᵢQKLHLCᵢᵢᵢ;
(SEQ ID NO: 42)
CᵢSAGWLTMCᵢᵢHKQLNLCᵢᵢᵢ;
(SEQ ID NO: 43)
CᵢS[Aib]GWLT[HPhe]CᵢᵢLKL[Aib]LCᵢᵢᵢ;
(SEQ ID NO: 44)
CᵢSAG[7-AzaW]LTMCᵢᵢQKLHLCᵢᵢᵢ;
(SEQ ID NO: 45)
CᵢSAGWLTMCᵢᵢ[Aad]KLHLCᵢᵢᵢ;
(SEQ ID NO: 46)
CᵢSAEWLTMCᵢᵢQKLHLCᵢᵢᵢ;
(SEQ ID NO: 47)
CᵢSA[Aad]WLTMCᵢᵢQKLHLCᵢᵢᵢ;
(SEQ ID NO: 48)
CᵢSAGWLTMCᵢᵢEKLHLCᵢᵢᵢ;
(SEQ ID NO: 49)
CᵢSAGWLTMCᵢᵢ[Cpap]KLHLCᵢᵢᵢ;
(SEQ ID NO: 50)
Cᵢ[AlloThr]AGWLTMCᵢᵢQKLHLCᵢᵢᵢ;
(SEQ ID NO: 51)
CᵢSAGW[Cpa]TMCᵢᵢQKLHLCᵢᵢᵢ;
(SEQ ID NO: 52)
CᵢSAGWLTMCᵢᵢQKLH[Cpa]Cᵢᵢᵢ;
(SEQ ID NO: 53)
CᵢS[Nle]GWLT[HPhe]CᵢᵢLKL[Aib]LCᵢᵢᵢ;
(SEQ ID NO: 54)
CᵢSEGWLTMCᵢᵢQKLHLCᵢᵢᵢ;
(SEQ ID NO: 55)
CᵢS[Aad]GWLTMCᵢᵢQKLHLCᵢᵢᵢ;
(SEQ ID NO: 56)
CᵢSDQWMQMCᵢᵢSKLTCᵢᵢᵢ;
(SEQ ID NO: 57)
CᵢSDGWLTMCᵢᵢQKLHLCᵢᵢᵢ;
(SEQ ID NO: 58)
CᵢSDEWLTMCᵢᵢQKLHLCᵢᵢᵢ;
(SEQ ID NO: 59)
CᵢSNSWLTMCᵢᵢQKLHLCᵢᵢᵢ;
(SEQ ID NO: 60)
CᵢSPAWLTMCᵢᵢQKLHLCᵢᵢᵢ;
(SEQ ID NO: 61)
CᵢSPEWLTMCᵢᵢQKLHLCᵢᵢᵢ;
(SEQ ID NO: 62)
CᵢSPGWLTMCᵢᵢQKLHLCᵢᵢᵢ;
(SEQ ID NO: 63)
CᵢSPQWLTMCᵢᵢQKLHLCᵢᵢᵢ;
(SEQ ID NO: 64)
CᵢSPSWLTMCᵢᵢQKLHLCᵢᵢᵢ;
(SEQ ID NO: 65)
CᵢSDSWKTMCᵢᵢQKLHLCᵢᵢᵢ;
(SEQ ID NO: 66)
CᵢSESWSTMCᵢᵢQKLHLCᵢᵢᵢ;
(SEQ ID NO: 67)
CᵢSPSWRTMCᵢᵢQKLHLCᵢᵢᵢ;
(SEQ ID NO: 68)
CᵢSPSWRNMCᵢᵢQKLHLCᵢᵢᵢ;
(SEQ ID NO: 69)
CᵢSWSWLTMCᵢᵢKQLNLCᵢᵢᵢ;
(SEQ ID NO: 70)
CᵢSWSWLTMCᵢᵢQKLDLCᵢᵢᵢ;
(SEQ ID NO: 71)
CᵢSSSWLTMCᵢᵢQKLHLCᵢᵢᵢ;
(SEQ ID NO: 72)
CᵢSWSWLNMCᵢᵢQKLHLCᵢᵢᵢ;
(SEQ ID NO: 73)
CᵢDPLCᵢᵢLSIRASLGLCᵢᵢᵢ;
(SEQ ID NO: 74)
CᵢDPLCᵢᵢLSIKRSLGLCᵢᵢᵢ;
(SEQ ID NO: 75)
CᵢDPLCᵢᵢLSIKRQLGLCᵢᵢᵢ;
(SEQ ID NO: 76)
CᵢDPLCᵢᵢLSIKRKLGLCᵢᵢᵢ;
(SEQ ID NO: 77)
CᵢDPLCᵢᵢLSIKRGLGLCᵢᵢᵢ;
(SEQ ID NO: 78)
CᵢDMRCᵢᵢIRIKQSLGMCᵢᵢᵢ;
(SEQ ID NO: 79)
CᵢRDWCᵢᵢHWTFDNGHPCᵢᵢᵢ;
(SEQ ID NO: 80)
CᵢRDWCᵢᵢHWTFSHGTPCᵢᵢᵢ;
(SEQ ID NO: 81)
CᵢRDWCᵢᵢHWTFSHGHPCᵢᵢᵢ;
(SEQ ID NO: 82)
CᵢRDWCᵢᵢHWTFTHGHPCᵢᵢᵢ;
(SEQ ID NO: 83)
CᵢRDWCᵢᵢHWTFTHSHPCᵢᵢᵢ;
(SEQ ID NO: 84)
CᵢS[Aad]GWLTMCᵢᵢQKLHLCᵢᵢᵢ;
(SEQ ID NO: 85)
CᵢS[Aad]GWLTMCᵢᵢLKLHLCᵢᵢᵢ;
(SEQ ID NO: 86)
CᵢS[Aad]GWL[3HyV]MCᵢᵢLKLHLCᵢᵢᵢ;
(SEQ ID NO: 87)
CᵢSKGWLTMCᵢᵢQ[K(Ac)]LHCᵢᵢᵢ;
(SEQ ID NO: 88)
CᵢSAGWLTKCᵢᵢQ[K(Ac)]LHLCᵢᵢᵢ;
(SEQ ID NO: 89)
CᵢSAGWLTMCᵢᵢK[K(Ac)]LHLCᵢᵢᵢ;
(SEQ ID NO: 90)
CᵢSAGWLTMCᵢᵢQ[K(Ac)]LKLCᵢᵢᵢ;
(SEQ ID NO: 91)
CᵢSAGWLTMCᵢᵢQ[HArg]LHLCᵢᵢᵢ;
(SEQ ID NO: 92)
CᵢSAGWLTMCᵢᵢ[HArg]QLNLCᵢᵢᵢ;
(SEQ ID NO: 93)
CᵢS[Aad]GWLTMCᵢᵢKQLNLCᵢᵢᵢ;
(SEQ ID NO: 94)
CᵢS[Aad]G[1Nal]LTMCᵢᵢKQLNLCᵢᵢᵢ;
(SEQ ID NO: 95)
[Pen]ᵢS[Aad]GWLTMCᵢᵢKQLNLCᵢᵢᵢ;
(SEQ ID NO: 96)
CᵢS[Aad]GWLTM[Pen]ᵢᵢKQLNLCᵢᵢᵢ;
and
(SEQ ID NO: 97)
CᵢS[Aad]GWLTMCᵢᵢKQLNL[Pen]ᵢᵢᵢ;

wherein Cᵢ, [Pen]ᵢ, Cᵢᵢ [Pen]ᵢᵢ, Cᵢᵢᵢ and [Pen]ᵢᵢᵢ represent first, second and third reactive groups, respectively, HArg represents homoarginine, HSer represents homoserine, HPhe represents homophenylalanine, Aib represents 2-aminoisobutyric acid, Abu represents 2-aminobutyric acid, 2Nal represents 3-(2-Naphthyl)-L-alanine, Chg represents Cyclohexylglycine, Nva represents Norvaline, 7-AzaW represents 7-azatryptophan, Aad represents 2-aminoadipic acid, Cpa represents β-cyclopropylalanine, Dab represents 2,4-diaminobutyric acid, 3HyV represents 2-amino-3-hydroxy-3-methyl-butyric acid, Ne represents norleucine, Pen represents penicillamine and PYA represents 4-pentynoic acid, or a pharmaceutically acceptable salt thereof.

In a further embodiment, the peptide ligand comprises an amino acid sequence selected from:
(B-Ala)-Sar5-A-(SEQ ID NO: 1)-A (herein referred to as 73-07-00-N001);
A-(SEQ ID NO: 1)-A (herein referred to as 73-07-00-N002 or BCY519);
A-(SEQ ID NO: 2)-A (herein referred to as 73-08-00-N002 or BCY521);
A-(SEQ ID NO: 3)-A (herein referred to as 73-09-00-N002 or BCY522);
A-(SEQ ID NO: 4)-A (herein referred to as 73-10-00-N002 or BCY523);
A-(SEQ ID NO: 5)-A (herein referred to as 73-13-00-N002 or BCY526).
A-(SEQ ID NO: 6)-A (herein referred to as 73-14-00-N002 or BCY527);
A-(SEQ ID NO: 7)-A (herein referred to as 73-14-01-N001 or BCY528);
A-(SEQ ID NO: 8)-A (herein referred to as 73-14-02-N001 or BCY529);
A-(SEQ ID NO: 9)-A (herein referred to as 73-14-03-N001 or BCY530);
A-(SEQ ID NO: 10)-A (herein referred to as 73-14-04-N001 or BCY531);
A-(SEQ ID NO: 11)-A (herein referred to as 73-14-05-N001 or BCY532);
A-(SEQ ID NO: 12)-A (herein referred to as 73-14-06-N001 or BCY533);
A-(SEQ ID NO: 13)-A (herein referred to as 73-14-07-N001 or BCY534);
A-(SEQ ID NO: 14)-A (herein referred to as 73-14-08-N001 or BCY535);
A-(SEQ ID NO: 15)-A (herein referred to as 73-14-10-N001 or BCY537);
A-(SEQ ID NO: 16)-A (herein referred to as 73-15-00-N002 or BCY538);
A-(SEQ ID NO: 17)-A (herein referred to as 73-16-00-N002 or BCY539);
[PYA]-[B-Ala]-[Sar₁₀]-(SEQ ID NO: 18) (hereinafter referred to as BCY8938);
SDK-(SEQ ID NO: 19)-A (hereinafter referred to as BCY3835);
[PYA]-[B-Ala]-[Sar₁₀]-SDK-(SEQ ID NO: 19) (hereinafter referred to as BCY10043);
NH₂—SDK-(SEQ ID NO: 19)-[Sar₁₀]-[K(PYA)](hereinafter referred to as BCY10044);
[Ac][dS]DK-(SEQ ID NO: 19)-A (hereinafter referred to as BCY10051);
SD[HArg]-(SEQ ID NO: 19)-A (hereinafter referred to as BCY10052);
SD[HSer]-(SEQ ID NO: 19)-A (hereinafter referred to as BCY10053);
[Ac]SDK-(SEQ ID NO: 19)-A (hereinafter referred to as BCY10075);
[Ac]SDR-(SEQ ID NO: 19) (hereinafter referred to as BCY10076);
[Ac]SDK-(SEQ ID NO: 19)-PSH (hereinafter referred to as BCY10943);
[Ac]SEK-(SEQ ID NO: 19) (hereinafter referred to as BCY10951);
[Ac]-SDK-(SEQ ID NO: 19) (hereinafter referred to as BCY11832);
[Ac]-A-(SEQ ID NO: 19)-PSH (hereinafter referred to as BCY11833);
SDK-(SEQ ID NO: 19)-A[Sar]₆ (the fluoresceinated derivative of which is hereinafter referred to as BCY566);
[Ac]D[HArg]-(SEQ ID NO: 19) (hereinafter referred to as BCY11865);
NH₂—SDK-(SEQ ID NO: 20) (hereinafter referred to as BCY10045);
Ac-SDK-(SEQ ID NO: 20)-PSH (hereinafter referred to as BCY10861);
Ac-SDK-(SEQ ID NO: 20) (hereinafter referred to as BCY11013);
SDK-(SEQ ID NO: 21)-A (hereinafter referred to as BCY10054);
SDK-(SEQ ID NO: 22)-A (hereinafter referred to as BCY10055);
SDK-(SEQ ID NO: 23)-A (hereinafter referred to as BCY10057);
SDK-(SEQ ID NO: 24)-A (hereinafter referred to as BCY10058);
SDK-(SEQ ID NO: 25)-A (hereinafter referred to as BCY10060);
SDK-(SEQ ID NO: 26)-A (hereinafter referred to as BCY10061);
SDK-(SEQ ID NO: 27)-A (hereinafter referred to as BCY10062);
SDK-(SEQ ID NO: 28)-A (hereinafter referred to as BCY10064);
SDK-(SEQ ID NO: 29)-A (hereinafter referred to as BCY10065);
SDK-(SEQ ID NO: 30)-A (hereinafter referred to as BCY10066);
SDK-(SEQ ID NO: 31)-A (hereinafter referred to as BCY10067);
[Ac]-SDK-(SEQ ID NO: 31)-PSH (hereinafter referred to as BCY11830);
SDK-(SEQ ID NO: 32)-A (hereinafter referred to as BCY10068);
SDK-(SEQ ID NO: 33)-A (hereinafter referred to as BCY10069);
SDK-(SEQ ID NO: 34)-A (hereinafter referred to as BCY10071);
SDK-(SEQ ID NO: 35)-A (hereinafter referred to as BCY10072);
SDK-(SEQ ID NO: 36)-A (hereinafter referred to as BCY10073);
SDK-(SEQ ID NO: 37)-A (hereinafter referred to as BCY10074);
[Ac]SDK-(SEQ ID NO: 38) (hereinafter referred to as BCY10078);
SDK-(SEQ ID NO: 39)-A (hereinafter referred to as BCY10083);
SDK-(SEQ ID NO: 40)-A (hereinafter referred to as BCY10084);
SDK-(SEQ ID NO: 41)-A (hereinafter referred to as BCY10085);
[Ac]SDK-(SEQ ID NO: 42)-PSH (hereinafter referred to as BCY10467);
[Ac]SDK-(SEQ ID NO: 42)-PS (hereinafter referred to as BCY10934);
[Ac]SDK-(SEQ ID NO: 42)-P (hereinafter referred to as BCY10935);
[Ac]SDK-(SEQ ID NO: 42)-PS—COOH (hereinafter referred to as BCY10936);
[Ac]SDK-(SEQ ID NO: 42)-P—COOH (hereinafter referred to as BCY10937);
[Ac]SDK-(SEQ ID NO: 42)-COH (hereinafter referred to as BCY10938);
[Ac]SDK-(SEQ ID NO: 42)-PSH—COOH (hereinafter referred to as BCY10939);
[Ac]DK-(SEQ ID NO: 42)-PSH (hereinafter referred to as BCY10940);
[Ac]K-(SEQ ID NO: 42)-PSH (hereinafter referred to as BCY10941);
[Ac]-(SEQ ID NO: 42)-PSH (hereinafter referred to as BCY10942);
[Ac]SD[HArg]-(SEQ ID NO: 43)-PSH (hereinafter referred to as BCY10944);
[Ac]SDK-(SEQ ID NO: 44) (hereinafter referred to as BCY10945);
[Ac]SDK-(SEQ ID NO: 45) (hereinafter referred to as BCY10946);
[Ac]SDK-(SEQ ID NO: 46) (hereinafter referred to as BCY10947);
[Ac]SDK-(SEQ ID NO: 47) (hereinafter referred to as BCY10948);
[Ac]SDK-(SEQ ID NO: 48) (hereinafter referred to as BCY10949);
[Ac]SDK-(SEQ ID NO: 49) (hereinafter referred to as BCY10950);
[Ac]SDK-(SEQ ID NO: 50) (hereinafter referred to as BCY10952);
[Ac]SDK-(SEQ ID NO: 51) (hereinafter referred to as BCY10954);
[Ac]SDK-(SEQ ID NO: 52) (hereinafter referred to as BCY10956);
[Ac]SD[HArg]-(SEQ ID NO: 53)-PSH (hereinafter referred to as BCY10959);
[Ac]SDK-(SEQ ID NO: 54) (hereinafter referred to as BCY10960);
[Ac]SDK-(SEQ ID NO: 55) (hereinafter referred to as BCY10961);
[B-Ala][Sar]₅A-(SEQ ID NO: 56)-A (the fluoresceinated derivative of which is hereinafter referred to as BCY562);
VER-(SEQ ID NO: 57)-A[Sar]₆ (the fluoresceinated derivative of which is hereinafter referred to as BCY567);
REN-(SEQ ID NO: 58)-A[Sar]₆ (the fluoresceinated derivative of which is hereinafter referred to as BCY568);
QQE-(SEQ ID NO: 59)-A[Sar]₆ (the fluoresceinated derivative of which is hereinafter referred to as BCY569);
SGK-(SEQ ID NO: 59)-A[Sar]₆ (the fluoresceinated derivative of which is hereinafter referred to as BCY577);
AGS-(SEQ ID NO: 60)-A[Sar]₆ (the fluoresceinated derivative of which is hereinafter referred to as BCY570);
AQT-(SEQ ID NO: 61)-A[Sar]₆ (the fluoresceinated derivative of which is hereinafter referred to as BCY571);
APV-(SEQ ID NO: 62)-A[Sar]₆ (the fluoresceinated derivative of which is hereinafter referred to as BCY572);
ADV-(SEQ ID NO: 63)-A[Sar]₆ (the fluoresceinated derivative of which is hereinafter referred to as BCY573);
GNK-(SEQ ID NO: 64)-A[Sar]₆ (the fluoresceinated derivative of which is hereinafter referred to as BCY574);
KPK-(SEQ ID NO: 64)-A[Sar]₆ (the fluoresceinated derivative of which is hereinafter referred to as BCY580);
ERV-(SEQ ID NO: 65)-A[Sar]₆ (the fluoresceinated derivative of which is hereinafter referred to as BCY575);
AER-(SEQ ID NO: 66)-A[Sar]₆ (the fluoresceinated derivative of which is hereinafter referred to as BCY576); KEL-(SEQ ID NO: 67)-A[Sar]₆ (the fluoresceinated derivative of which is hereinafter referred to as BCY578);
G[Sar]₅KEL-(SEQ ID NO: 67)-A (the fluoresceinated derivative of which is hereinafter referred to as BCY3811);
KEL-(SEQ ID NO: 68)-A[Sar]₆ (the fluoresceinated derivative of which is hereinafter referred to as BCY579);
A-(SEQ ID NO: 69)-PSH[Sar]₆ (the fluoresceinated derivative of which is hereinafter referred to as BCY581);
A-(SEQ ID NO: 70)-DHEN[Sar]₆ (the fluoresceinated derivative of which is hereinafter referred to as BCY582);
REE-(SEQ ID NO: 71)-A[Sar]₆ (the fluoresceinated derivative of which is hereinafter referred to as BCY3812);
QAEK-(SEQ ID NO: 72)-A[Sar]₆ (the fluoresceinated derivative of which is hereinafter referred to as BCY8210);
A-(SEQ ID NO: 73)-A[Sar]₆ (the fluoresceinated derivative of which is hereinafter referred to as BCY7126);
A-(SEQ ID NO: 74)-A[Sar]₆ (the fluoresceinated derivative of which is hereinafter referred to as BCY7374);
A-(SEQ ID NO: 75)-A[Sar]₆ (the fluoresceinated derivative of which is hereinafter referred to as BCY7375);
A-(SEQ ID NO: 76)-A[Sar]₆ (the fluoresceinated derivative of which is hereinafter referred to as BCY7376);
A-(SEQ ID NO: 77)-A[Sar]₆ (the fluoresceinated derivative of which is hereinafter referred to as BCY7377);
A-(SEQ ID NO: 78)-A[Sar]₆ (the fluoresceinated derivative of which is hereinafter referred to as BCY7384);
A-(SEQ ID NO: 79)-A[Sar]₆ (the fluoresceinated derivative of which is hereinafter referred to as BCY7385);
A-(SEQ ID NO: 80)-A[Sar]₆ (the fluoresceinated derivative of which is hereinafter referred to as BCY7386);
A-(SEQ ID NO: 81)-A[Sar]₆ (the fluoresceinated derivative of which is hereinafter referred to as BCY7387);
A-(SEQ ID NO: 82)-A[Sar]₆ (the fluoresceinated derivative of which is hereinafter referred to as BCY7388);
A-(SEQ ID NO: 83)-A[Sar]₆ (the fluoresceinated derivative of which is hereinafter referred to as BCY7389);
Ac-DK-(SEQ ID NO: 84) (hereinafter referred to as BCY11818);
Ac-K-(SEQ ID NO: 84) (hereinafter referred to as BCY11819);
Ac-SEK-(SEQ ID NO: 84) (hereinafter referred to as BCY11820);
Ac-EK-(SEQ ID NO: 84) (hereinafter referred to as BCY11821);
Ac-DK-(SEQ ID NO: 84)-PSH—COOH (hereinafter referred to as BCY11853);
Ac-SDK-(SEQ ID NO: 85) (hereinafter referred to as BCY11822);
Ac-DK-(SEQ ID NO: 85) (hereinafter referred to as BCY11823);
Ac-K-(SEQ ID NO: 85) (hereinafter referred to as BCY11824);
Ac-SEK-(SEQ ID NO: 85) (hereinafter referred to as BCY11825);
Ac-EK-(SEQ ID NO: 85) (hereinafter referred to as BCY11826);
Ac-[HArg]-(SEQ ID NO: 85) (hereinafter referred to as BCY11827);
Ac-SDK-(SEQ ID NO: 85)-A (hereinafter referred to as BCY11828);
Ac-SEK-(SEQ ID NO: 85)-PSH (hereinafter referred to as BCY11831);
Ac-SDK-(SEQ ID NO: 86) (hereinafter referred to as BCY11829);
Ac-D[HArg]-(SEQ ID NO: 87)-PSH (hereinafter referred to as BCY11866);
Ac-D[HArg]-(SEQ ID NO: 88)-PSH (hereinafter referred to as BCY11867);
Ac-D[HArg]-(SEQ ID NO: 89)-PSH (hereinafter referred to as BCY11868);
Ac-D[HArg]-(SEQ ID NO: 90)-PSH (hereinafter referred to as BCY11869);
Ac-SD[HArg]-(SEQ ID NO: 91)-PSHK (hereinafter referred to as BCY12479);
Ac-SD[HArg]-(SEQ ID NO: 92)-PSHK (hereinafter referred to as BCY12477); Ac-[HArg]-(SEQ ID NO: 93)-PSH (hereinafter referred to as BCY12640);
Ac-[HArg]-(SEQ ID NO: 94)-PSH (hereinafter referred to as BCY12641);
Ac-[HArg]-(SEQ ID NO: 95)-PSH (hereinafter referred to as BCY12642);
Ac-[HArg]-(SEQ ID NO: 96)-PSH (hereinafter referred to as BCY12643); and
Ac-[HArg]-(SEQ ID NO: 97)-PSH (hereinafter referred to as BCY12644);
   wherein PYA represents 4-pentynoic acid, B-Ala represents beta-alanine, Sar represents sarcosine, HSer represents homoserine and HArg represents homoarginine, or a pharmaceutically acceptable salt thereof.

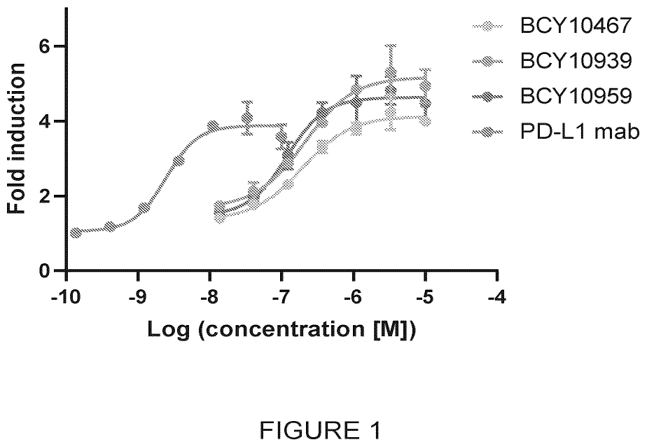

In one embodiment, the peptide ligand is a peptide sequence which is selected from any one of: BCY10467, BCY10939 and BCY10959. Data is presented herein in FIG. 1 and Table 6 where it can be seen that these PD-L1 binding bicyclic peptides demonstrated the ability to block PD-1/PD-L1 interaction between PD-1 expressing T cells and CHO-K1 stable expressing PD-L1.

In one embodiment, the peptide ligand is selected from a peptide sequence which is other than any one or more of: BCY11818, BCY11819, BCY11820, BCY11821, BCY11822, BCY11823, BCY11824, BCY11825, BCY11826, BCY11827, BCY11828, BCY11829, BCY11830, BCY11831, BCY11832, BCY11833 and/or BCY11853.

In one embodiment, the molecular scaffold is selected from 1,1′,1″-(1,3,5-triazinane-1,3,5-triyl)triprop-2-en-1-one (TATA).

In one embodiment, the peptide ligand is selected from a peptide listed in any of Tables 1 to 5.

Unless defined otherwise, all technical and scientific terms used herein have the same meaning as commonly understood by those of ordinary skill in the art, such as in the arts of peptide chemistry, cell culture and phage display, nucleic acid chemistry and biochemistry. Standard techniques are used for molecular biology, genetic and biochemical methods (see Sambrook et al., Molecular Cloning: A Laboratory Manual, 3rd ed., 2001, Cold Spring Harbor Laboratory Press, Cold Spring Harbor, NY; Ausubel et al., Short Protocols in Molecular Biology (1999) 4^{th} ed., John Wiley & Sons, Inc.), which are incorporated herein by reference.

### Nomenclature

### Numbering

When referring to amino acid residue positions within the peptides of the invention, cysteine residues (Cᵢ, Cᵢᵢ and Cᵢᵢᵢ) are omitted from the numbering as they are invariant, therefore, the numbering of amino acid residues within the peptides of the invention is referred to as below:

(SEQ ID NO: 1)
-Cᵢ-S₁-W₂-S₃-W₄-L₅-T₆-M₇-Cᵢᵢ-Q₈-K₉-L₁₀-H₁₁-L₁₂-Cᵢᵢᵢ.

For the purpose of this description, all bicyclic peptides are assumed to be cyclised with, 1′,1″-(1,3,5-triazinane-1,3,5-triyl)triprop-2-en-1-one (TATA) and yielding a tri-substituted structure. Cyclisation with TATA occurs on Cᵢ, Cᵢᵢ, and Cᵢᵢᵢ.

### Molecular Format

N- or C-terminal extensions to the bicycle core sequence are added to the left or right side of the sequence, separated by a hyphen. For example, an N-terminal βAla-Sar₁₀-Ala tail would be denoted as:

(SEQ ID NO: X)
βAla-Sar10-A-.

### Inversed Peptide Sequences

In light of the disclosure in Nair et al(2003) J Immunol 170(3), 1362-1373, it is envisaged that the peptide sequences disclosed herein would also find utility in their retro-inverso form. For example, the sequence is reversed (i.e. N-terminus becomes C-terminus and vice versa) and their stereochemistry is likewise also reversed (i.e. D-amino acids become L-amino acids and vice versa).

### Peptide Ligands

A peptide ligand, as referred to herein, refers to a peptide covalently bound to a molecular scaffold. Typically, such peptides comprise two or more reactive groups (i.e. cysteine and/or Pen residues) which are capable of forming covalent bonds to the scaffold, and a sequence subtended between said reactive groups which is referred to as the loop sequence, since it forms a loop when the peptide is bound to the scaffold. In the present case, the peptides either comprise at least three cysteine residues (referred to herein as Cᵢ, Cᵢᵢ and Cᵢᵢᵢ) or one penicillamine residue and two cysteine residues, and form at least two loops on the scaffold.

### Reactive Groups

The molecular scaffold of the invention may be bonded to the polypeptide via functional or reactive groups on the polypeptide. These are typically formed from the side chains of particular amino acids found in the polypeptide polymer. Such reactive groups may be a cysteine side chain, a lysine side chain, or an N-terminal amine group or any other suitable reactive group, such as penicillamine. Details of suitable reactive groups may be found in WO 2009/098450.

Examples of reactive groups of natural amino acids are the thiol group of cysteine, the amino group of lysine, the carboxyl group of aspartate or glutamate, the guanidinium group of arginine, the phenolic group of tyrosine or the hydroxyl group of serine. Non-natural amino acids can provide a wide range of reactive groups including an azide, a keto-carbonyl, an alkyne, a vinyl, or an aryl halide group. The amino and carboxyl group of the termini of the polypeptide can also serve as reactive groups to form covalent bonds to a molecular scaffold/molecular core.

The polypeptides of the invention contain at least three reactive groups. Said polypeptides can also contain four or more reactive groups. The more reactive groups are used, the more loops can be formed in the molecular scaffold.

In a preferred embodiment, polypeptides with three reactive groups are generated. Reaction of said polypeptides with a molecular scaffold/molecular core having a three-fold rotational symmetry generates a single product isomer. The generation of a single product isomer is favourable for several reasons. The nucleic acids of the compound libraries encode only the primary sequences of the polypeptide but not the isomeric state of the molecules that are formed upon reaction of the polypeptide with the molecular core. If only one product isomer can be formed, the assignment of the nucleic acid to the product isomer is clearly defined. If multiple product isomers are formed, the nucleic acid cannot give information about the nature of the product isomer that was isolated in a screening or selection process. The formation of a single product isomer is also advantageous if a specific member of a library of the invention is synthesized. In this case, the chemical reaction of the polypeptide with the molecular scaffold yields a single product isomer rather than a mixture of isomers.

In another embodiment, polypeptides with four reactive groups are generated. Reaction of said polypeptides with a molecular scaffold/molecular core having a tetrahedral symmetry generates two product isomers. Even though the two different product isomers are encoded by one and the same nucleic acid, the isomeric nature of the isolated isomer can be determined by chemically synthesizing both isomers, separating the two isomers and testing both isomers for binding to a target ligand.

In one embodiment of the invention, at least one of the reactive groups of the polypeptides is orthogonal to the remaining reactive groups. The use of orthogonal reactive groups allows the directing of said orthogonal reactive groups to specific sites of the molecular core. Linking strategies involving orthogonal reactive groups may be used to limit the number of product isomers formed. In other words, by choosing distinct or different reactive groups for one or more of the at least three bonds to those chosen for the remainder of the at least three bonds, a particular order of bonding or directing of specific reactive groups of the polypeptide to specific positions on the molecular scaffold may be usefully achieved.

In another embodiment, the reactive groups of the polypeptide of the invention are reacted with molecular linkers wherein said linkers are capable to react with a molecular scaffold so that the linker will intervene between the molecular scaffold and the polypeptide in the final bonded state.

In some embodiments, amino acids of the members of the libraries or sets of polypeptides can be replaced by any natural or non-natural amino acid. Excluded from these exchangeable amino acids are the ones harbouring functional groups for cross-linking the polypeptides to a molecular core, such that the loop sequences alone are exchangeable.

The exchangeable polypeptide sequences have either random sequences, constant sequences or sequences with random and constant amino acids. The amino acids with reactive groups are either located in defined positions within the polypeptide, since the position of these amino acids determines loop size.

In one embodiment, a polypeptide with three reactive groups has the sequence (X)ₗY(X)ₘY(X)ₙY(X)ₒ, wherein Y represents an amino acid with a reactive group, X represents a random amino acid, m and n are numbers between 3 and 6 defining the length of intervening polypeptide segments, which may be the same or different, and l and o are numbers between 0 and 20 defining the length of flanking polypeptide segments.

Alternatives to thiol-mediated conjugations can be used to attach the molecular scaffold to the peptide via covalent interactions. Alternatively these techniques may be used in modification or attachment of further moieties (such as small molecules of interest which are distinct from the molecular scaffold) to the polypeptide after they have been selected or isolated according to the present invention—in this embodiment then clearly the attachment need not be covalent and may embrace non-covalent attachment. These methods may be used instead of (or in combination with) the thiol mediated methods by producing phage that display proteins and peptides bearing unnatural amino acids with the requisite chemical reactive groups, in combination small molecules that bear the complementary reactive group, or by incorporating the unnatural amino acids into a chemically or recombinantly synthesised polypeptide when the molecule is being made after the selection/isolation phase. Further details can be found in WO 2009/098450 or Heinis et al., Nat Chem Biol 2009, 5 (7), 502-7.

In one embodiment, the reactive groups are selected from cysteine and/or penicillamine:

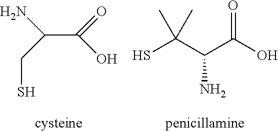

In one embodiment, each of said reactive groups comprise cysteine. In an alternative, embodiment one of said reactive groups comprises penicillamine and the remaining (i.e. two) reactive groups comprise cysteine.

### Advantages of the Peptide Ligands

Certain bicyclic peptides of the present invention have a number of advantageous properties which enable them to be considered as suitable drug-like molecules for injection, inhalation, nasal, ocular, oral or topical administration. Such advantageous properties include:
Species cross-reactivity. This is a typical requirement for preclinical pharmacodynamics and pharmacokinetic evaluation;
Protease stability. Bicyclic peptide ligands should ideally demonstrate stability to plasma proteases, epithelial (“membrane-anchored”) proteases, gastric and intestinal proteases, lung surface proteases, intracellular proteases and the like. Protease stability should be maintained between different species such that a bicycle lead candidate can be developed in animal models as well as administered with confidence to humans;
Desirable solubility profile. This is a function of the proportion of charged and hydrophilic versus hydrophobic residues and intra/inter-molecular H-bonding, which is important for formulation and absorption purposes;
An optimal plasma half-life in the circulation. Depending upon the clinical indication and treatment regimen, it may be required to develop a bicyclic peptide for short exposure in an acute illness management setting, or develop a bicyclic peptide with enhanced retention in the circulation, and is therefore optimal for the management of more chronic disease states. Other factors driving the desirable plasma half-life are requirements of sustained exposure for maximal therapeutic efficiency versus the accompanying toxicology due to sustained exposure of the agent; and
Selectivity. Certain peptide ligands of the invention demonstrate good selectivity over other transmembrane proteins.

### Pharmaceutically Acceptable Salts

It will be appreciated that salt forms are within the scope of this invention, and references to peptide ligands include the salt forms of said ligands.

The salts of the present invention can be synthesized from the parent compound that contains a basic or acidic moiety by conventional chemical methods such as methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002. Generally, such salts can be prepared by reacting the free acid or base forms of these compounds with the appropriate base or acid in water or in an organic solvent, or in a mixture of the two.

Acid addition salts (mono- or di-salts) may be formed with a wide variety of acids, both inorganic and organic. Examples of acid addition salts include mono- or di-salts formed with an acid selected from the group consisting of acetic, 2,2-dichloroacetic, adipic, alginic, ascorbic (e.g. L-ascorbic), L-aspartic, benzenesulfonic, benzoic, 4-acetamidobenzoic, butanoic, (+) camphoric, camphor-sulfonic, (+)-(1 S)-camphor-10-sulfonic, capric, caproic, caprylic, cinnamic, citric, cyclamic, dodecylsulfuric, ethane-1,2-disulfonic, ethanesulfonic, 2-hydroxyethanesulfonic, formic, fumaric, galactaric, gentisic, glucoheptonic, D-gluconic, glucuronic (e.g. D-glucuronic), glutamic (e.g. L-glutamic), a-oxoglutaric, glycolic, hippuric, hydrohalic acids (e.g. hydrobromic, hydrochloric, hydriodic), isethionic, lactic (e.g. (+)-L-lactic, (±)-DL-lactic), lactobionic, maleic, malic, (−)-L-malic, malonic, (±)-DL-mandelic, methanesulfonic, naphthalene-2-sulfonic, naphthalene-1,5-disulfonic, 1-hydroxy-2-naphthoic, nicotinic, nitric, oleic, orotic, oxalic, palmitic, pamoic, phosphoric, propionic, pyruvic, L-pyroglutamic, salicylic, 4-amino-salicylic, sebacic, stearic, succinic, sulfuric, tannic, (+)-L-tartaric, thiocyanic, p-toluenesulfonic, undecylenic and valeric acids, as well as acylated amino acids and cation exchange resins.

One particular group of salts consists of salts formed from acetic, hydrochloric, hydriodic, phosphoric, nitric, sulfuric, citric, lactic, succinic, maleic, malic, isethionic, fumaric, benzenesulfonic, toluenesulfonic, sulfuric, methanesulfonic (mesylate), ethanesulfonic, naphthalenesulfonic, valeric, propanoic, butanoic, malonic, glucuronic and lactobionic acids. One particular salt is the hydrochloride salt. Another particular salt is the acetate salt.

If the compound is anionic, or has a functional group which may be anionic (e.g., —COOH may be —COO⁻), then a salt may be formed with an organic or inorganic base, generating a suitable cation. Examples of suitable inorganic cations include, but are not limited to, alkali metal ions such as Li⁺, Na⁺ and K⁺, alkaline earth metal cations such as Ca²⁺ and Mg²⁺, and other cations such as Al³⁺ or Zn⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: methylamine, ethylamine, diethylamine, propylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺. Where the peptides of the invention contain an amine function, these may form quaternary ammonium salts, for example by reaction with an alkylating agent according to methods well known to the skilled person. Such quaternary ammonium compounds are within the scope of peptides of the invention.

### Modified Derivatives

It will be appreciated that modified derivatives of the peptide ligands as defined herein are within the scope of the present invention. Examples of such suitable modified derivatives include one or more modifications selected from: N-terminal and/or C-terminal modifications; replacement of one or more amino acid residues with one or more non-natural amino acid residues (such as replacement of one or more polar amino acid residues with one or more isosteric or isoelectronic amino acids; replacement of one or more non-polar amino acid residues with other non-natural isosteric or isoelectronic amino acids); addition of a spacer group; replacement of one or more oxidation sensitive amino acid residues with one or more oxidation resistant amino acid residues; replacement of one or more amino acid residues with an alanine, replacement of one or more L-amino acid residues with one or more D-amino acid residues; N-alkylation of one or more amide bonds within the bicyclic peptide ligand; replacement of one or more peptide bonds with a surrogate bond; peptide backbone length modification; substitution of the hydrogen on the alpha-carbon of one or more amino acid residues with another chemical group, modification of amino acids such as cysteine, lysine, glutamate/aspartate and tyrosine with suitable amine, thiol, carboxylic acid and phenol-reactive reagents so as to functionalise said amino acids, and introduction or replacement of amino acids that introduce orthogonal reactivities that are suitable for functionalisation, for example azide or alkyne-group bearing amino acids that allow functionalisation with alkyne or azide-bearing moieties, respectively.

In one embodiment, the modified derivative comprises an N-terminal and/or C-terminal modification. In a further embodiment, wherein the modified derivative comprises an N-terminal modification using suitable amino-reactive chemistry, and/or C-terminal modification using suitable carboxy-reactive chemistry. In a further embodiment, said N-terminal or C-terminal modification comprises addition of an effector group, including but not limited to a cytotoxic agent, a radiochelator or a chromophore.

In a further embodiment, the modified derivative comprises an N-terminal modification. In a further embodiment, the N-terminal modification comprises an N-terminal acetyl group. In this embodiment, the N-terminal cysteine group (the group referred to herein as Cᵢ) is capped with acetic anhydride or other appropriate reagents during peptide synthesis leading to a molecule which is N-terminally acetylated. This embodiment provides the advantage of removing a potential recognition point for aminopeptidases and avoids the potential for degradation of the bicyclic peptide.

In an alternative embodiment, the N-terminal modification comprises the addition of a molecular spacer group which facilitates the conjugation of effector groups and retention of potency of the bicyclic peptide to its target.

In a further embodiment, the modified derivative comprises a C-terminal modification. In a further embodiment, the C-terminal modification comprises an amide group. In this embodiment, the C-terminal cysteine group (the group referred to herein as Cᵢᵢᵢ) is synthesized as an amide during peptide synthesis leading to a molecule which is C-terminally amidated. This embodiment provides the advantage of removing a potential recognition point for carboxypeptidase and reduces the potential for proteolytic degradation of the bicyclic peptide.

In one embodiment, the modified derivative comprises replacement of one or more amino acid residues with one or more non-natural amino acid residues. In this embodiment, non-natural amino acids may be selected having isosteric/isoelectronic side chains which are neither recognised by degradative proteases nor have any adverse effect upon target potency.

Alternatively, non-natural amino acids may be used having constrained amino acid side chains, such that proteolytic hydrolysis of the nearby peptide bond is conformationally and sterically impeded. In particular, these concern proline analogues, bulky sidechains, Ca-disubstituted derivatives (for example, aminoisobutyric acid, Aib), and cyclo amino acids, a simple derivative being amino-cyclopropylcarboxylic acid.

In one embodiment, the modified derivative comprises the addition of a spacer group. In a further embodiment, the modified derivative comprises the addition of a spacer group to the N-terminal cysteine (Cᵢ) and/or the C-terminal cysteine (Cᵢᵢᵢ).

In one embodiment, the modified derivative comprises replacement of one or more oxidation sensitive amino acid residues with one or more oxidation resistant amino acid residues. In a further embodiment, the modified derivative comprises replacement of a tryptophan residue with a naphthylalanine or alanine residue. This embodiment provides the advantage of improving the pharmaceutical stability profile of the resultant bicyclic peptide ligand.

In one embodiment, the modified derivative comprises replacement of one or more charged amino acid residues with one or more hydrophobic amino acid residues. In an alternative embodiment, the modified derivative comprises replacement of one or more hydrophobic amino acid residues with one or more charged amino acid residues. The correct balance of charged versus hydrophobic amino acid residues is an important characteristic of the bicyclic peptide ligands. For example, hydrophobic amino acid residues influence the degree of plasma protein binding and thus the concentration of the free available fraction in plasma, while charged amino acid residues (in particular arginine) may influence the interaction of the peptide with the phospholipid membranes on cell surfaces. The two in combination may influence half-life, volume of distribution and exposure of the peptide drug, and can be tailored according to the clinical endpoint. In addition, the correct combination and number of charged versus hydrophobic amino acid residues may reduce irritation at the injection site (if the peptide drug has been administered subcutaneously).

In one embodiment, the modified derivative comprises replacement of one or more L-amino acid residues with one or more D-amino acid residues. This embodiment is believed to increase proteolytic stability by steric hindrance and by a propensity of D-amino acids to stabilise P-turn conformations (Tugyi et al(2005) PNAS, 102(2), 413-418).

In one embodiment, the modified derivative comprises removal of any amino acid residues and substitution with alanines. This embodiment provides the advantage of removing potential proteolytic attack site(s).

It should be noted that each of the above mentioned modifications serve to deliberately improve the potency or stability of the peptide. Further potency improvements based on modifications may be achieved through the following mechanisms:
Incorporating hydrophobic moieties that exploit the hydrophobic effect and lead to lower off rates, such that higher affinities are achieved;
Incorporating charged groups that exploit long-range ionic interactions, leading to faster on rates and to higher affinities (see for example Schreiber et al, Rapid, electrostatically assisted association of proteins (1996), Nature Struct. Biol. 3, 427-31); and
Incorporating additional constraint into the peptide, by for example constraining side chains of amino acids correctly such that loss in entropy is minimal upon target binding, constraining the torsional angles of the backbone such that loss in entropy is minimal upon target binding and introducing additional cyclisations in the molecule for identical reasons.
   (for reviews see Gentilucci et al, Curr. Pharmaceutical Design, (2010), 16, 3185-203, and Nestor et al, Curr. Medicinal Chem (2009), 16, 4399-418).

### Isotopic Variations

The present invention includes all pharmaceutically acceptable (radio)isotope-labeled peptide ligands of the invention, wherein one or more atoms are replaced by atoms having the same atomic number, but an atomic mass or mass number different from the atomic mass or mass number usually found in nature, and peptide ligands of the invention, wherein metal chelating groups are attached (termed “effector”) that are capable of holding relevant (radio)isotopes, and peptide ligands of the invention, wherein certain functional groups are covalently replaced with relevant (radio)isotopes or isotopically labelled functional groups.

Examples of isotopes suitable for inclusion in the peptide ligands of the invention comprise isotopes of hydrogen, such as ²H (D) and ³H (T), carbon, such as C, ¹³C and ¹⁴C, chlorine, such as ³⁶Cl, fluorine, such as ¹⁸F, iodine, such as ¹²³I, ¹²⁵I and ¹³¹I, nitrogen, such as ¹³N and ¹⁵N, oxygen, such as ¹⁵O, ¹⁷O and ¹⁸O, phosphorus, such as ³²P, sulfur, such as ³⁵S, copper, such as ⁶⁴Cu, gallium, such as ⁶⁷Ga or ⁶⁸Ga, yttrium, such as ⁹⁰Y and lutetium, such as ¹⁷⁷Lu, and Bismuth, such as ²¹³Bi.

Certain isotopically-labelled peptide ligands of the invention, for example, those incorporating a radioactive isotope, are useful in drug and/or substrate tissue distribution studies, and to clinically assess the presence and/or absence of the PD-L1 target on diseased tissues. The peptide ligands of the invention can further have valuable diagnostic properties in that they can be used for detecting or identifying the formation of a complex between a labelled compound and other molecules, peptides, proteins, enzymes or receptors. The detecting or identifying methods can use compounds that are labelled with labelling agents such as radioisotopes, enzymes, fluorescent substances, luminous substances (for example, luminol, luminol derivatives, luciferin, aequorin and luciferase), etc. The radioactive isotopes tritium, i.e. ³H (T), and carbon-14, i.e. ¹⁴C, are particularly useful for this purpose in view of their ease of incorporation and ready means of detection.

Substitution with heavier isotopes such as deuterium, i.e. ²H (D), may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining target occupancy.

Isotopically-labelled compounds of peptide ligands of the invention can generally be prepared by conventional techniques known to those skilled in the art or by processes analogous to those described in the accompanying Examples using an appropriate isotopically-labelled reagent in place of the non-labelled reagent previously employed.

Non-Aromatic Molecular scaffold

References herein to the term “non-aromatic molecular scaffold” refer to any molecular scaffold as defined herein which does not contain an aromatic (i.e. unsaturated) carbocyclic or heterocyclic ring system.

Suitable examples of non-aromatic molecular scaffolds are described in Heinis et al (2014) Angewandte Chemie, International Edition 53(6) 1602-1606.

As noted in the foregoing documents, the molecular scaffold may be a small molecule, such as a small organic molecule.

In one embodiment the molecular scaffold may be a macromolecule. In one embodiment the molecular scaffold is a macromolecule composed of amino acids, nucleotides or carbohydrates.

In one embodiment the molecular scaffold comprises reactive groups that are capable of reacting with functional group(s) of the polypeptide to form covalent bonds.

The molecular scaffold may comprise chemical groups which form the linkage with a peptide, such as amines, thiols, alcohols, ketones, aldehydes, nitriles, carboxylic acids, esters, alkenes, alkynes, azides, anhydrides, succinimides, maleimides, alkyl halides and acyl halides.

In one embodiment, the molecular scaffold may comprise or may consist of 1,1′,1″-(1,3,5-triazinane-1,3,5-triyl)triprop-2-en-1-one (TATA), or a derivative thereof.

An example of an us unsaturated carbonyl containing compound is 1,1′,1″-(1,3,5-triazinane-1,3,5-triyl)triprop-2-en-1-one (TATA) (Angewandte Chemie, International Edition (2014), 53(6), 1602-1606).

### Effector and Functional Groups

According to a further aspect of the invention, there is provided a drug conjugate comprising a peptide ligand as defined herein conjugated to one or more effector and/or functional groups.

Effector and/or functional groups can be attached, for example, to the N and/or C termini of the polypeptide, to an amino acid within the polypeptide, or to the molecular scaffold.

Appropriate effector groups include antibodies and parts or fragments thereof. For instance, an effector group can include an antibody light chain constant region (CL), an antibody CH1 heavy chain domain, an antibody CH2 heavy chain domain, an antibody CH3 heavy chain domain, or any combination thereof, in addition to the one or more constant region domains. An effector group may also comprise a hinge region of an antibody (such a region normally being found between the CH1 and CH2 domains of an IgG molecule).

In a further embodiment of this aspect of the invention, an effector group according to the present invention is an Fc region of an IgG molecule. Advantageously, a peptide ligand-effector group according to the present invention comprises or consists of a peptide ligand Fc fusion having a tβ half-life of a day or more, two days or more, 3 days or more, 4 days or more, 5 days or more, 6 days or more or 7 days or more. Most advantageously, the peptide ligand according to the present invention comprises or consists of a peptide ligand Fc fusion having a tβ half-life of a day or more.

Functional groups include, in general, binding groups, drugs, reactive groups for the attachment of other entities, functional groups which aid uptake of the macrocyclic peptides into cells, and the like.

The ability of peptides to penetrate into cells will allow peptides against intracellular targets to be effective. Targets that can be accessed by peptides with the ability to penetrate into cells include transcription factors, intracellular signalling molecules such as tyrosine kinases and molecules involved in the apoptotic pathway. Functional groups which enable the penetration of cells include peptides or chemical groups which have been added either to the peptide or the molecular scaffold. Peptides such as those derived from such as VP22, HIV-Tat, a homeobox protein of Drosophila (Antennapedia), e.g. as described in Chen and Harrison, Biochemical Society Transactions (2007) Volume 35, part 4, p 821; Gupta et al. in Advanced Drug Discovery Reviews (2004) Volume 57 9637. Examples of short peptides which have been shown to be efficient at translocation through plasma membranes include the 16 amino acid penetratin peptide from Drosophila Antennapedia protein (Derossi et al (1994) J Biol. Chem. Volume 269 p 10444), the 18 amino acid ‘model amphipathic peptide’ (Oehlke et al (1998) Biochim Biophys Acts Volume 1414 p 127) and arginine rich regions of the HIV TAT protein. Non peptidic approaches include the use of small molecule mimics or SMOCs that can be easily attached to biomolecules (Okuyama et al (2007) Nature Methods Volume 4 p 153). Other chemical strategies to add guanidinium groups to molecules also enhance cell penetration (Elson-Scwab et al (2007) J Biol Chem Volume 282 p 13585). Small molecular weight molecules such as steroids may be added to the molecular scaffold to enhance uptake into cells.

One class of functional groups which may be attached to peptide ligands includes antibodies and binding fragments thereof, such as Fab, Fv or single domain fragments. In particular, antibodies which bind to proteins capable of increasing the half-life of the peptide ligand in vivo may be used.

In one embodiment, a peptide ligand-effector group according to the invention has a tβ half-life selected from the group consisting of: 12 hours or more, 24 hours or more, 2 days or more, 3 days or more, 4 days or more, 5 days or more, 6 days or more, 7 days or more, 8 days or more, 9 days or more, 10 days or more, 11 days or more, 12 days or more, 13 days or more, 14 days or more, 15 days or more or 20 days or more. Advantageously a peptide ligand-effector group or composition according to the invention will have a tβ half-life in the range 12 to 60 hours. In a further embodiment, it will have a tβ half-life of a day or more. In a further embodiment still, it will be in the range 12 to 26 hours.

In one particular embodiment of the invention, the functional group is selected from a metal chelator, which is suitable for complexing metal radioisotopes of medicinal relevance.

Possible effector groups also include enzymes, for instance such as carboxypeptidase G2 for use in enzyme/prodrug therapy, where the peptide ligand replaces antibodies in ADEPT.

In one particular embodiment of the invention, the functional group is selected from a drug, such as a cytotoxic agent for cancer therapy. Suitable examples include: alkylating agents such as cisplatin and carboplatin, as well as oxaliplatin, mechlorethamine, cyclophosphamide, chlorambucil, ifosfamide; Anti-metabolites including purine analogs azathioprine and mercaptopurine or pyrimidine analogs; plant alkaloids and terpenoids including vinca alkaloids such as Vincristine, Vinblastine, Vinorelbine and Vindesine; Podophyllotoxin and its derivatives etoposide and teniposide; Taxanes, including paclitaxel, originally known as Taxol; topoisomerase inhibitors including camptothecins: irinotecan and topotecan, and type II inhibitors including amsacrine, etoposide, etoposide phosphate, and teniposide. Further agents can include antitumour antibiotics which include the immunosuppressant dactinomycin (which is used in kidney transplantations), doxorubicin, epirubicin, bleomycin, calicheamycins, and others.

In one further particular embodiment of the invention, the cytotoxic agent is selected from maytansinoids (such as DM1) or monomethyl auristatins (such as MMAE).

DM1 is a cytotoxic agent which is a thiol-containing derivative of maytansine and has the following structure:

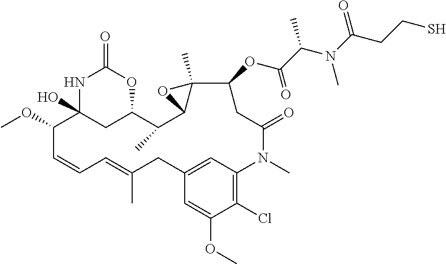 [SMILES: COc1cc2cc(c1Cl)N(C)C(=O)C[C@H](OC(=O)[C@H](C)N(C)C(=O)CCS)[C@]1(C)O[C@H]1[C@H](C)[C@@H]1C[C@@](O)(NC(=O)O1)[C@@H](OC)/C=C\C=C(/C)C2]

Monomethyl auristatin E (MMAE) is a synthetic antineoplastic agent and has the following structure:

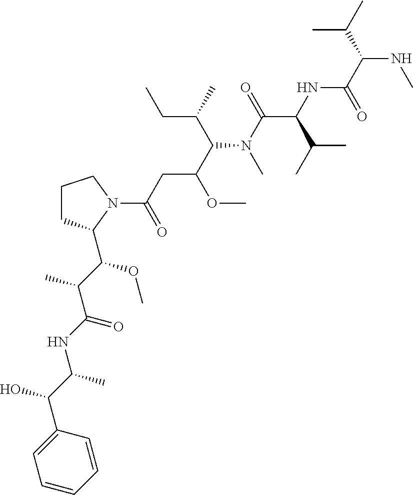

In one yet further particular embodiment of the invention, the cytotoxic agent is selected from maytansinoids (such as DM1).

In one embodiment, the cytotoxic agent is linked to the bicyclic peptide by a cleavable bond, such as a disulphide bond or a protease sensitive bond. In a further embodiment, the groups adjacent to the disulphide bond are modified to control the hindrance of the disulphide bond, and by this the rate of cleavage and concomitant release of cytotoxic agent.

Published work established the potential for modifying the susceptibility of the disulphide bond to reduction by introducing steric hindrance on either side of the disulphide bond (Kellogg et al (2011) Bioconjugate Chemistry, 22, 717). A greater degree of steric hindrance reduces the rate of reduction by intracellular glutathione and also extracellular (systemic) reducing agents, consequentially reducing the ease by which toxin is released, both inside and outside the cell. Thus, selection of the optimum in disulphide stability in the circulation (which minimises undesirable side effects of the toxin) versus efficient release in the intracellular milieu (which maximises the therapeutic effect) can be achieved by careful selection of the degree of hindrance on either side of the disulphide bond.

The hindrance on either side of the disulphide bond is modulated through introducing one or more methyl groups on either the targeting entity (here, the bicyclic peptide) or toxin side of the molecular construct.

In one embodiment, the cytotoxic agent and linker is selected from any combinations of those described in WO 2016/067035 (the cytotoxic agents and linkers thereof are herein incorporated by reference).

### Synthesis

The peptides of the present invention may be manufactured synthetically by standard techniques followed by reaction with a molecular scaffold in vitro. When this is performed, standard chemistry may be used. This enables the rapid large scale preparation of soluble material for further downstream experiments or validation. Such methods could be accomplished using conventional chemistry such as that disclosed in Timmerman et al (supra).

Thus, the invention also relates to manufacture of polypeptides or conjugates selected as set out herein, wherein the manufacture comprises optional further steps as explained below. In one embodiment, these steps are carried out on the end product polypeptide/conjugate made by chemical synthesis.

Optionally amino acid residues in the polypeptide of interest may be substituted when manufacturing a conjugate or complex.

Peptides can also be extended, to incorporate for example another loop and therefore introduce multiple specificities.

To extend the peptide, it may simply be extended chemically at its N-terminus or C-terminus or within the loops using orthogonally protected lysines (and analogues) using standard solid phase or solution phase chemistry. Standard (bio)conjugation techniques may be used to introduce an activated or activatable N- or C-terminus. Alternatively additions may be made by fragment condensation or native chemical ligation e.g. as described in (Dawson et al. 1994. Synthesis of Proteins by Native Chemical Ligation. Science 266:776-779), or by enzymes, for example using subtiligase as described in (Chang et al Proc Natl Acad Sci USA. 1994 Dec. 20; 91(26):12544-8 or in Hikari et al Bioorganic & Medicinal Chemistry Letters Volume 18, Issue 22, 15 Nov. 2008, Pages 6000-6003).

Alternatively, the peptides may be extended or modified by further conjugation through disulphide bonds. This has the additional advantage of allowing the first and second peptide to dissociate from each other once within the reducing environment of the cell. In this case, the molecular scaffold could be added during the chemical synthesis of the first peptide so as to react with the three cysteine groups; a further cysteine or thiol could then be appended to the N or C-terminus of the first peptide, so that this cysteine or thiol only reacted with a free cysteine or thiol of the second peptide, forming a disulfide-linked bicyclic peptide-peptide conjugate.

Similar techniques apply equally to the synthesis/coupling of two bicyclic and bispecific macrocycles, potentially creating a tetraspecific molecule.

Furthermore, addition of other functional groups or effector groups may be accomplished in the same manner, using appropriate chemistry, coupling at the N- or C-termini or via side chains. In one embodiment, the coupling is conducted in such a manner that it does not block the activity of either entity.

### Pharmaceutical Compositions

According to a further aspect of the invention, there is provided a pharmaceutical composition comprising a peptide ligand or a drug conjugate as defined herein in combination with one or more pharmaceutically acceptable excipients.

Generally, the present peptide ligands will be utilised in purified form together with pharmacologically appropriate excipients or carriers. Typically, these excipients or carriers include aqueous or alcoholic/aqueous solutions, emulsions or suspensions, including saline and/or buffered media. Parenteral vehicles include sodium chloride solution, Ringer's dextrose, dextrose and sodium chloride and lactated Ringer's. Suitable physiologically-acceptable adjuvants, if necessary to keep a polypeptide complex in suspension, may be chosen from thickeners such as carboxymethylcellulose, polyvinylpyrrolidone, gelatin and alginates.

Intravenous vehicles include fluid and nutrient replenishers and electrolyte replenishers, such as those based on Ringer's dextrose. Preservatives and other additives, such as antimicrobials, antioxidants, chelating agents and inert gases, may also be present (Mack (1982) Remington's Pharmaceutical Sciences, 16th Edition).

The peptide ligands of the present invention may be used as separately administered compositions or in conjunction with other agents. These can include antibodies, antibody fragments and various immunotherapeutic drugs, such as cylcosporine, methotrexate, adriamycin or cisplatinum and immunotoxins. Pharmaceutical compositions can include “cocktails” of various cytotoxic or other agents in conjunction with the protein ligands of the present invention, or even combinations of selected polypeptides according to the present invention having different specificities, such as polypeptides selected using different target ligands, whether or not they are pooled prior to administration.

The route of administration of pharmaceutical compositions according to the invention may be any of those commonly known to those of ordinary skill in the art. For therapy, the peptide ligands of the invention can be administered to any patient in accordance with standard techniques. The administration can be by any appropriate mode, including parenterally, intravenously, intramuscularly, intraperitoneally, transdermally, via the pulmonary route, or also, appropriately, by direct infusion with a catheter. Preferably, the pharmaceutical compositions according to the invention will be administered by inhalation. The dosage and frequency of administration will depend on the age, sex and condition of the patient, concurrent administration of other drugs, counterindications and other parameters to be taken into account by the clinician.

The peptide ligands of this invention can be lyophilised for storage and reconstituted in a suitable carrier prior to use. This technique has been shown to be effective and art-known lyophilisation and reconstitution techniques can be employed. It will be appreciated by those skilled in the art that lyophilisation and reconstitution can lead to varying degrees of activity loss and that levels may have to be adjusted upward to compensate.

The compositions containing the present peptide ligands or a cocktail thereof can be administered for prophylactic and/or therapeutic treatments. In certain therapeutic applications, an adequate amount to accomplish at least partial inhibition, suppression, modulation, killing, or some other measurable parameter, of a population of selected cells is defined as a “therapeutically-effective dose”. Amounts needed to achieve this dosage will depend upon the severity of the disease and the general state of the patient's own immune system, but generally range from 0.005 to 5.0 mg of selected peptide ligand per kilogram of body weight, with doses of 0.05 to 2.0 mg/kg/dose being more commonly used. For prophylactic applications, compositions containing the present peptide ligands or cocktails thereof may also be administered in similar or slightly lower dosages.

A composition containing a peptide ligand according to the present invention may be utilised in prophylactic and therapeutic settings to aid in the alteration, inactivation, killing or removal of a select target cell population in a mammal. In addition, the peptide ligands described herein may be used extracorporeally or in vitro selectively to kill, deplete or otherwise effectively remove a target cell population from a heterogeneous collection of cells. Blood from a mammal may be combined extracorporeally with the selected peptide ligands whereby the undesired cells are killed or otherwise removed from the blood for return to the mammal in accordance with standard techniques.

### Therapeutic Uses

The bicyclic peptides of the invention have specific utility as PD-L1 binding agents.

Programmed cell death 1 ligand 1 (PD-L1) is a 290 amino acid type I transmembrane protein encoded by the CD274 gene on mouse chromosome 19 and human chromosome 9. PD-L1 expression is involved in evasion of immune responses involved in chronic infection, e.g., chronic viral infection (including, for example, HIV, HBV, HCV and HTLV, among others), chronic bacterial infection (including, for example, Helicobacter pylori, among others), and chronic parasitic infection (including, for example, Schistosoma mansoni). PD-L1 expression has been detected in a number of tissues and cell types including T-cells, B-cells, macrophages, dendritic cells, and nonhaematopoietic cells including endothelial cells, hepatocytes, muscle cells, and placenta.

PD-L1 expression is also involved in suppression of anti-tumour immune activity. Tumours express antigens that can be recognised by host T-cells, but immunologic clearance of tumours is rare. Part of this failure is due to immune suppression by the tumour microenvironment. PD-L1 expression on many tumours is a component of this suppressive milieu and acts in concert with other immunosuppressive signals. PD-L1 expression has been shown in situ on a wide variety of solid tumours including breast, lung, colon, ovarian, melanoma, bladder, liver, salivary, stomach, gliomas, thyroid, thymic epithelial, head, and neck (Brown J A et al. 2003 Immunol. 170:1257-66; Dong H et al. 2002 Nat. Med. 8:793-800; Hamanishi J, et al. 2007 Proc. Natl. Acad. Sci. USA 104:3360-65; Strome S E et al. 2003 Cancer Res. 63:6501-5; Inman B A et al. 2007 Cancer 109:1499-505; Konishi J et al. 2004 Clin. Cancer Res. 10:5094-100; Nakanishi J et al. 2007 Cancer Immunol. Immunother. 56:1173-82; Nomi T et al. 2007 Clin. Cancer Res. 13:2151-57; Thompson R H et al. 2004 Proc. Natl. Acad. Sci. USA 101: 17174-79; Wu C et al. 2006 Acta Histochem. 108:19-24). In addition, the expression of the receptor for PD-L1, Programmed cell death protein 1 (also known as PD-1 and CD279) is upregulated on tumour infiltrating lymphocytes, and this also contributes to tumour immunosuppression (Blank C et al. 2003 Immunol. 171:4574-81). Most importantly, studies relating PD-L1 expression on tumours to disease outcome show that PD-L1 expression strongly correlates with unfavourable prognosis in kidney, ovarian, bladder, breast, gastric, and pancreatic cancer (Hamanishi J et al. 2007 Proc. Natl. Acad. Sci. USA 104:3360-65; Inman B A et al. 2007 Cancer 109:1499-505; Konishi J et al. 2004 Clin. Cancer Res. 10:5094-100; Nakanishi J et al. 2007 Cancer Immunol. Immunother. 56:1173-82; Nomi T et al. 2007 Clin. Cancer Res. 13:2151-57; Thompson R H et al. 2004 Proc. Natl. Acad. Sci. USA 101:17174-79; Wu C et al. 2006 Acta Histochem. 108:19-24). In addition, these studies suggest that higher levels of PD-L1 expression on tumours may facilitate advancement of tumour stage and invasion into deeper tissue structures.

The PD-1 pathway can also play a role in haematologic malignancies. PD-L1 is expressed on multiple myeloma cells but not on normal plasma cells (Liu J et al. 2007 Blood 110:296-304). PD-L1 is expressed on some primary T-cell lymphomas, particularly anaplastic large cell T lymphomas (Brown J A et al, 2003 Immunol. 170:1257-66). PD-1 is highly expressed on the T-cells of angioimmunoblastic lymphomas, and PD-L1 is expressed on the associated follicular dendritic cell network (Dorfman D M et al. 2006 Am. J. Surg. Pathol. 30:802-10). In nodular lymphocyte-predominant Hodgkin lymphoma, the T-cells associated with lymphocytic or histiocytic (L&H) cells express PD-1. Microarray analysis using a readout of genes induced by PD-1 ligation suggests that tumour-associated T-cells are responding to PD-1 signals in situ in Hodgkin lymphoma (Chemnitz J M et al. 2007 Blood 110:3226-33). PD-1 and PD-L1 are expressed on CD4 T-cells in HTLV-1-mediated adult T-cell leukaemia and lymphoma (Shimauchi T et al. 2007 Int. J. Cancer 121: 2585-90). These tumour cells are hyporesponsive to TCR signals.

Studies in animal models demonstrate that PD-L1 on tumours inhibits T-cell activation and lysis of tumour cells and in some cases leads to increased tumour-specific T-cell death (Dong H et al. 2002 Nat. Med. 8:793-800; Hirano F et al. 2005 Cancer Res. 65:1089-96). Tumour-associated APCs can also utilise the PD-1:PD-L1 pathway to control antitumour T-cell responses. PD-L1 expression on a population of tumour-associated myeloid DCs is upregulated by tumour environmental factors (Curiel T J et al. 2003 Nat. Med. 9:562-67). Plasmacytoid dendritic cells (DCs) in the tumour-draining lymph node of B16 melanoma express IDO, which strongly activates the suppressive activity of regulatory T-cells. The suppressive activity of IDO-treated regulatory T-cells required cell contact with IDO-expressing DCs (Sharma M D et al. 2007 Clin. Invest. 117:2570-82).

Accordingly, there is a need in the art for effective treatments for PD-L1-associated diseases, such as an infectious disease, such as a chronic intracellular infectious disease, e.g., a viral disease, e.g., hepatitis infection, or a bacterial infection, e.g., tuberculosis infection; and cancer, e.g., a hepatic cancer, e.g., hepatocellular carcinoma.

Polypeptide ligands selected according to the method of the present invention may be employed in in vivo therapeutic and prophylactic applications, in vitro and in vivo diagnostic applications, in vitro assay and reagent applications, and the like. Ligands having selected levels of specificity are useful in applications which involve testing in non-human animals, where cross-reactivity is desirable, or in diagnostic applications, where cross-reactivity with homologues or paralogues needs to be carefully controlled. In some applications, such as vaccine applications, the ability to elicit an immune response to predetermined ranges of antigens can be exploited to tailor a vaccine to specific diseases and pathogens.

Substantially pure peptide ligands of at least 90 to 95% homogeneity are preferred for administration to a mammal, and 98 to 99% or more homogeneity is most preferred for pharmaceutical uses, especially when the mammal is a human. Once purified, partially or to homogeneity as desired, the selected polypeptides may be used diagnostically or therapeutically (including extracorporeally) or in developing and performing assay procedures, immunofluorescent stainings and the like (Lefkovite and Pernis, (1979 and 1981) Immunological Methods, Volumes I and II, Academic Press, NY).

According to a further aspect of the invention, there is provided a peptide ligand or a drug conjugate as defined herein, for use in preventing, suppressing or treating a disease or disorder mediated by PD-L1.

According to a further aspect of the invention, there is provided a method of preventing, suppressing or treating a disease or disorder mediated by PD-L1, which comprises administering to a patient in need thereof an effector group and drug conjugate of the peptide ligand as defined herein.

In one embodiment, the PD-L1 is mammalian PD-L1. In a further embodiment, the mammalian PD-L1 is human PD-L1 (hPD-L1) or mouse PD-L1 (mPD-L1). All peptide sequences defined herein are human PD-L1 binding sequences with the exception of BCY8938 which binds to mouse PD-L1. In a further embodiment, the mammalian PD-L1 is human PD-L1 (hPD-L1).

In one embodiment, the disease or disorder mediated by PD-L1 is selected from chronic infection or disease, solid tumours and haematologic malignancies.

In one embodiment, the chronic infection or disease mediated by PD-L1 is selected from chronic viral infection, chronic bacterial infection, chronic parasitic infection, hepatitis infection, and viral disease.

In one embodiment, the solid tumour mediated by PD-L1 is selected from breast, lung, colon, ovarian, melanoma, bladder, liver, salivary, stomach, gliomas, thyroid, thymic epithelial, head, and neck tumour In a further embodiment, the disease or disorder mediated by PD-L1 is selected from cancer.

Examples of cancers (and their benign counterparts) which may be treated (or inhibited) include, but are not limited to tumours of epithelial origin (adenomas and carcinomas of various types including adenocarcinomas, squamous carcinomas, transitional cell carcinomas and other carcinomas) such as carcinomas of the bladder and urinary tract, breast, gastrointestinal tract (including the esophagus, stomach (gastric), small intestine, colon, rectum and anus), liver (hepatocellular carcinoma), gall bladder and biliary system, exocrine pancreas, kidney, lung (for example adenocarcinomas, small cell lung carcinomas, non-small cell lung carcinomas, bronchioalveolar carcinomas and mesotheliomas), head and neck (for example cancers of the tongue, buccal cavity, larynx, pharynx, nasopharynx, tonsil, salivary glands, nasal cavity and paranasal sinuses), ovary, fallopian tubes, peritoneum, vagina, vulva, penis, cervix, myometrium, endometrium, thyroid (for example thyroid follicular carcinoma), adrenal, prostate, skin and adnexae (for example melanoma, basal cell carcinoma, squamous cell carcinoma, keratoacanthoma, dysplastic naevus); haematological malignancies (i.e. leukemias, lymphomas) and premalignant haematological disorders and disorders of borderline malignancy including haematological malignancies and related conditions of lymphoid lineage (for example acute lymphocytic leukemia [ALL], chronic lymphocytic leukemia [CLL], B-cell lymphomas such as diffuse large B-cell lymphoma [DLBCL], follicular lymphoma, Burkitt's lymphoma, mantle cell lymphoma, T-cell lymphomas and leukaemias, natural killer [NK]cell lymphomas, Hodgkin's lymphomas, hairy cell leukaemia, monoclonal gammopathy of uncertain significance, plasmacytoma, multiple myeloma, and post-transplant lymphoproliferative disorders), and haematological malignancies and related conditions of myeloid lineage (for example acute myelogenousleukemia [AML], chronic myelogenousleukemia [CML], chronic myelomonocyticleukemia [CMML], hypereosinophilic syndrome, myeloproliferative disorders such as polycythaemia vera, essential thrombocythaemia and primary myelofibrosis, myeloproliferative syndrome, myelodysplastic syndrome, and promyelocyticleukemia); tumours of mesenchymal origin, for example sarcomas of soft tissue, bone or cartilage such as osteosarcomas, fibrosarcomas, chondrosarcomas, rhabdomyosarcomas, leiomyosarcomas, liposarcomas, angiosarcomas, Kaposi's sarcoma, Ewing's sarcoma, synovial sarcomas, epithelioid sarcomas, gastrointestinal stromal tumours, benign and malignant histiocytomas, and dermatofibrosarcomaprotuberans; tumours of the central or peripheral nervous system (for example astrocytomas, gliomas and glioblastomas, meningiomas, ependymomas, pineal tumours and schwannomas); endocrine tumours (for example pituitary tumours, adrenal tumours, islet cell tumours, parathyroid tumours, carcinoid tumours and medullary carcinoma of the thyroid); ocular and adnexal tumours (for example retinoblastoma); germ cell and trophoblastic tumours (for example teratomas, seminomas, dysgerminomas, hydatidiform moles and choriocarcinomas); and paediatric and embryonal tumours (for example medulloblastoma, neuroblastoma, Wilms tumour, and primitive neuroectodermal tumours); or syndromes, congenital or otherwise, which leave the patient susceptible to malignancy (for example Xeroderma Pigmentosum).

In a further embodiment, the cancer is a cancer of the kidney, ovary, bladder, breast, gastric, and pancreas.

In a yet further embodiment, the cancer is selected from a liver cancer e.g., hepatic cancer and hepatocellular carcinoma.

In a further embodiment, the cancer is selected from a hematopoietic malignancy such as selected from: non-Hodgkin's lymphoma (NHL), Burkitt's lymphoma (BL), multiple myeloma (MM), B chronic lymphocytic leukemia (B-CLL), B and T acute lymphocytic leukemia (ALL), T cell lymphoma (TCL), acute myeloid leukemia (AML), hairy cell leukemia (HCL), Hodgkin's Lymphoma (HL), chronic myeloid leukemia (CML), and nodular lymphocyte-predominant Hodgkin lymphoma.

References herein to the term “prevention” involves administration of the protective composition prior to the induction of the disease. “Suppression” refers to administration of the composition after an inductive event, but prior to the clinical appearance of the disease. “Treatment” involves administration of the protective composition after disease symptoms become manifest.

Animal model systems which can be used to screen the effectiveness of the peptide ligands in protecting against or treating the disease are available. The use of animal model systems is facilitated by the present invention, which allows the development of polypeptide ligands which can cross react with human and animal targets, to allow the use of animal models.

The invention is further described below with reference to the following examples.

### EXAMPLES

### Materials and Methods

### Peptide Synthesis

Peptide synthesis was based on Fmoc chemistry, using a Symphony peptide synthesiser manufactured by Peptide Instruments and a Syro II synthesiser by MultiSynTech. Standard Fmoc-amino acids were employed (Sigma, Merck), with appropriate side chain protecting groups: where applicable standard coupling conditions were used in each case, followed by deprotection using standard methodology. Peptides were purified using HPLC and following isolation they were modified with 1,3,5-Triacryloylhexahydro-1,3,5-triazine (TATA, Sigma). For this, linear peptide was diluted with 50:50 MeCN:H₂O up to ˜35 mL, ˜500 μL of 100 mM TATA in acetonitrile was added, and the reaction was initiated with 5 mL of 1 M NHaHCO₃ in H₂O. The reaction was allowed to proceed for ˜30 −60 min at RT, and lyophilised once the reaction had completed (judged by MALDI). Once completed, 1 ml of 1M L-cysteine hydrochloride monohydrate (Sigma) in H₂O was added to the reaction for ˜60 min at RT to quench any excess TATA. Following lyophilisation, the modified peptide was purified as above, while replacing the Luna C8 with a Gemini C18 column (Phenomenex), and changing the acid to 0.1% trifluoroacetic acid. Pure fractions containing the correct TATA-modified material were pooled, lyophilised and kept at −20° C. for storage.

All amino acids, unless noted otherwise, were used in the L-configurations.

In some cases peptides are converted to activated disulfides prior to coupling with the free thiol group of a toxin using the following method; a solution of 4-methyl(succinimidyl 4-(2-pyridylthio)pentanoate) (100 mM) in dry DMSO (1.25 mol equiv) was added to a solution of peptide (20 mM) in dry DMSO (1 mol equiv). The reaction was well mixed and DIPEA (20 mol equiv) was added. The reaction was monitored by LC/MS until complete.

### Biological Data

### 1. PD-L1 Direct Binding Assay

Affinity of the peptides of the invention for human PD-L1 (Ki) was determined using a fluorescence polarisation assay, in accordance with the methods disclosed in WO2016/067035. Peptides of the invention with a fluorescent tag (either fluorescein, SIGMA or Alexa Fluor488™, Fisher Scientific) were diluted to 2.5 nM in PBS with 0.01% tween 20 or 50 mM HEPES with 100 mM NaCl and 0.01% tween pH 7.4 (both referred to as assay buffer). This was combined with a titration of protein in the same assay buffer as the peptide to give 1 nM peptide in a total volume of 25 μL in a black walled and bottomed low bind low volume 384 well plates, typically 5 μL assay buffer, 10 μL protein then 10 μL fluorescent peptide. One in two serial dilutions were used to give 12 different concentrations with top concentrations ranging from 500 nM for known high affinity binders to 10 μM for low affinity binders and selectivity assays. Measurements were conducted on a BMG PHERAstar FS equipped with an “FP 485 520 520” optic module which excites at 485 nm and detects parallel and perpendicular emission at 520 nm. The PHERAstar FS was set at 25° C. with 200 flashes per well and a positioning delay of 0.1 second, with each well measured at 5 to 10 minute intervals for 60 minutes. The gain used for analysis was determined for each tracer at the end of the 60 minutes where there was no protein in the well. Data was analysed using Systat Sigmaplot version 12.0. mP values were fit to a user defined quadratic equation to generate a Kd value: f=ymin+(ymax−ymin)/Lig*((x+Lig+Kd)/2−sqrt((((x+Lig+Kd)/2)\hat{ }2)−(Lig*x))). “Lig” was a defined value of the concentration of tracer used.

The following selected bicyclic peptide ligands of the invention were tested in the above mentioned PD-Li Direct Binding Assay and the results are shown in Tables 1 and 2:

TABLE 1
Human Direct Binding Assay Data for Selected Bicyclic Peptide Ligands of the
Invention

| | | Molecular | |
|---|---|---|---|
| Peptide Number | Sequence | Scaffold | Kd (nM) |
| 73-07-00-N001 | FI-(B-Ala)-Sar₅-ACSWSWLTMCQKLHLCA | TATA | 16.9 |
| | (FI-(B-Ala)-Sar₅-BCY519) | | |
| | (FI-(B-Ala)-Sar5-A-(SEQ ID NO: 1)-A) | | |
| 73-07-00-N002 | ACSWSWLTMCQKLHLCA | TATA | 29.6 |
| (BCY519) | (A-(SEQ ID NO: 1)-A) | | |
| 73-08-00-N002 | ACSTSWMNLCKQLNLCA | TATA | 170 |
| (BCY521) | (A-(SEQ ID NO: 2)-A) | | |
| 73-09-00-N002 | ACSPIWTNTCIQLGLCA | TATA | 618 |
| (BCY522) | (A-(SEQ ID NO: 3)-A) | | |
| 73-10-00-N002 | ACTPNWNAMCLKLNLCA | TATA | 362 |
| (BCY523) | (A-(SEQ ID NO: 4)-A) | | |
| 73-13-00-N002 | ACDVFTHCILLAKPCA | TATA | 378.75 |
| (BCY526) | (A-(SEQ ID NO: 5)-A) | | |
| 73-14-00-N002 | ACSESWSNMCVSLGLCA | TATA | 493.75* |
| (BCY527) | (A-(SEQ ID NO: 6)-A) | | |
| 73-14-01-N001 | ACSAEWRNMCVQLDLCA | TATA | 161.5* |
| (BCY528) | (A-(SEQ ID NO: 7)-A) | | |
| 73-14-02-N001 | ACSASWSNMCVELGLCA | TATA | 356.15* |
| (BCY529) | (A-(SEQ ID NO: 8)-A) | | |
| 73-14-03-N001 | ACSDNWLNMCVELGLCA | TATA | 106.45 |
| (BCY530) | (A-(SEQ ID NO: 9)-A) | | |
| 73-14-04-N001 | ACSESWSAMCASLGLCA | TATA | 311.03* |
| (BCY531) | (A-(SEQ ID NO: 10)-A) | | |
| 73-14-05-N001 | ACSESWSNMCKSLGLCA | TATA | 175.5* |
| (BCY532) | (A-(SEQ ID NO: 11)-A) | | |
| 73-14-06-N001 | ACSESWRNMCVQLNLCA | TATA | 19.2 |
| (BCY533) | (A-(SEQ ID NO: 12)-A) | | |
| 73-14-07-N001 | ACSPEVVTNMCVQLHLCA | TATA | 65.75 |
| (BCY534) | (A-(SEQ ID NO: 13)-A) | | |
| 73-14-08-N001 | ACSTQWNNMCVQLGLCA | TATA | 115.65 |
| (BCY535) | (A-(SEQ ID NO: 14)-A) | | |
| 73-14-10-N001 | ACSSSVVTNMCVQLGLCA | TATA | 49.45 |
| (BCY537) | (A-(SEQ ID NO: 15)-A) | | |
| 73-15-00-N002 | ACSAEWRNMCVELNLCA | TATA | 524 |
| (BCY538) | (A-(SEQ ID NO: 16)-A) | | |
| 73-16-00-N002 | ACSPEWKNMCITLNLCA | TATA | 116 |
| (BCY539) | (A-(SEQ ID NO: 17)-A) | | |
| BCY552 | [FI][B-Ala][Sar]₅ACSWSWLTMCQKLHLCA | TATA | 121.3 ± 58.83 |
| | ([FI][B-Ala][Sar]₅-BCY519) | | |
| | ([FI][B-Ala][Sar]₅-A-(SEQ ID NO: 1)-A) | | |
| BCY553 | [FI][B-Ala][Sar]₅ACSTSWMNLCKQLNLCA | TATA | 494.57 ± 367.92 |
| | ([FI][B-Ala][Sar]₅-BCY521) | | |
| | ([FI][B-Ala][Sar]₅-A-(SEQ ID NO: 2)-A) | | |
| BCY555 | [FI][B-Ala][Sar]₅ACTPNWNAMCLKLNLCA | TATA | 748 ± 150.92 |
| | ([FI][B-Ala][Sar]₅-BCY523) | | |
| | ([FI][B-Ala][Sar]₅-A-(SEQ ID NO: 4)-A) | | |
| BCY559 | [FI][B-Ala][Sar]₅ACSESWSNMCVSLGLCA | TATA | 438.1 ± 190.41 |
| | ([FI][B-Ala][Sar]₅-BCY527) | | |
| | ([FI][B-Ala][Sar]₅-A-(SEQ ID NO: 6)-A) | | |
| BCY560 | [FI][B-Ala][Sar]₅ACSAEWRNMCVELNLCA | TATA | 491.83 ± 353 |
| | ([FI][B-Ala][Sar]₅-BCY538) | | |
| | ([FI][B-Ala][SaR]₅-A-(SEQ ID NO: 16)-A) | | |
| BCY561 | [FI][B-Ala][Sar]₅ACSPEWKNMCITLNLCA | TATA | 365.03 ± 232.83 |
| | ([FI][B-Ala][Sar]₅-BCY539) | | |
| | ([FI][B-Ala][Sar]₅-A-(SEQ ID NO: 17)-A) | | |
| BCY562 | [FI][B-Ala][Sar]₅ACSDQWMQMCSKLTCA | TATA | 934.72 |
| | ([FI][B-Ala][Sar]₅A-(SEQ ID NO: 56)-A) | | |
| BCY566 | SDKCSAGWLTMCQKLHLCA[Sar]₆[KFI] | TATA | 12.62 ± 3.35 |
| | (SDK-(SEQ ID NO: 19)-A[Sar]₆[KFI]) | | |
| BCY567 | VERCSDGWLTMCQKLHLCA[Sar]₆[KFI] | TATA | 16.4 ± 3.84 |
| | (VER-(SEQ ID NO: 57)-A[Sar]₆[KFI]) | | |
| BCY568 | RENCSDEWLTMCQKLHLCA[Sar]₆[KFI] | TATA | 78.96 ± 32.24 |
| | (REN-(SEQ ID NO: 58)-A[Sar]₆[KFI]) | | |
| BCY569 | QQECSNSWLTMCQKLHLCA[Sar]₆[KFI] | TATA | 83.94 ± 26.71 |
| | (QQE-(SEQ ID NO: 59)-A[Sar]₆[KFI]) | | |
| BCY570 | AGSCSPAWLTMCQKLHLCA[Sar]₆[KFI] | TATA | 80.18 ± 14.08 |
| | (AGS-(SEQ ID NO: 60)-A[Sar]₆[KFI]) | | |
| BCY571 | AQTCSPEWLTMCQKLHLCA[Sar]₆[KFI] | TATA | 125 ± 39.61 |
| | (AQT-(SEQ ID NO: 61)-A[Sar]₆[KFI]) | | |
| BCY572 | APVCSPGWLTMCQKLHLCA[Sar]₆[KFI] | TATA | 45.53 ± 5.07 |
| | (APV-(SEQ ID NO: 62)-A[Sar]₆[KFI]) | | |
| BCY573 | ADVCSPQWLTMCQKLHLCA[Sar]₆[KFI] | TATA | 30.17 ± 12.85 |
| | (ADV-(SEQ ID NO: 63)-A[Sar]₆[KFI]) | | |
| BCY574 | GNKCSPSWLTMCQKLHLCA[Sar]₆[KFI] | TATA | 16.93 ± 4.68 |
| | (GNK-(SEQ ID NO: 64)-A[Sar]₆[KFI]) | | |
| BCY575 | ERVCSDSWKTMCQKLHLCA[Sar]₆[KFI] | TATA | 134.33 ± 7.95 |
| | (ERV-(SEQ ID NO: 65)-A[Sar]₆[KFI]) | | |
| BCY576 | AERCSESWSTMCQKLHLCA[Sar]₆[KFI] | TATA | 124 ± 63.09 |
| | (AER-(SEQ ID NO: 66)-A[Sar]₆[KFI]) | | |
| BCY577 | SGKCSNSWLTMCQKLHLCA[Sar]₆[KFI] | TATA | 51.5 ± 26.71 |
| | (SGK-(SEQ ID NO: 59)-A[Sar]₆[KFI]) | | |
| BCY578 | KELCSPSWRTMCQKLHLCA[Sar]₆[KFI] | TATA | 55.1 ± 19.35 |
| | (KEL-(SEQ ID NO: 67)-A[Sar]₆[KFI]) | | |
| BCY579 | KELCSPSWRNMCQKLHLCA[Sar]₆[KFI] | TATA | 58.6 ± 47.82 |
| | (KEL-(SEQ ID NO: 68)-A[Sar]₆[KFI]) | | |
| BCY580 | KPKCSPSWLTMCQKLHLCA[Sar]₆[KFI] | TATA | 50.95 ± 49.1 |
| | (KPK-(SEQ ID NO: 64)-A[Sar]₆[KFI]) | | |
| BCY581 | ACSWSWLTMCKQLNLCPSH[Sar]₆[KFI](A- | TATA | 24.25 ± 27.24 |
| | (SEQ ID NO: 69)-PSH[Sar]₆[KFI]) | | |
| BCY582 | ACSWSWLTMCQKLDLCDHEN[Sar]₆[KFI] | TATA | 176.5 ± 36.26 |
| | (A-(SEQ ID NO: 70)-DHEN[Sar]₆[KFI]) | | |
| BCY3808 | [FI][B-Ala][Sar]₅ACSESWRNMCVQLNLCA | TATA | 43.65 ± 0.88 |
| | ([FI][B-Ala][Sar]₅-BCY533) | | |
| | ([FI][B-Ala][Sar]₅-A-(SEQ ID NO: 12)-A) | | |
| BCY3809 | [FI]G[Sar]₅ACSSSWTNMCVQLGLCA | TATA | 57.35 ± 6.37 |
| | ([FI]G[Sar]₅-BCY537) ([FI]G[Sar]₅-A-(SEQ | | |
| | ID NO: 15)-A) | | |
| BCY3810 | ACSSSWTNMCVQLGLCA[Sar]₆[KFI] | TATA | 32.9 ± 0.39 |
| | (BCY537-[Sar]₆[KFI]) | | |
| | (A-(SEQ ID NO: 15)-A-[Sar]₆[KFI]) | | |
| BCY3811 | [FI]G[Sar]₅KELCSPSWRTMCQKLHLCA | TATA | 54.85 ± 8.13 |
| | ([FI]G[Sar]₅KEL-(SEQ ID NO: 67)-A) | | |
| BCY3812 | REECSSSWLTMCQKLHLCA[Sar]₆[KFI] | TATA | 65.35 ± 4.8 |
| | (REE-(SEQ ID NO: 71)-A[Sar]₆[KFI]) | | |
| BCY8210 | QAEKCSWSWLNMCQKLHLCA[Sar]₆[KFI] | TATA | 9.97 ± 1.03 |
| | (QAEK-(SEQ ID NO: 72)-A[Sar]₆[KFI]) | | |

TABLE 2
Mouse Direct Binding Assay Data for Selected Bicyclic
Peptide Ligands of the Invention

| Peptide | | | Kd |
|---|---|---|---|
| Number | Sequence | Scaffold | (nM) |
| BCY7126 | ACDPLCLSIRASLGLCA[Sar]₆[KFI] | TATA | 943 |
| | (A-(SEQ ID NO: 73)-A[Sar]₆[KFI]) | | |
| BCY7374 | ACDPLCLSIKRSLGLCA[Sar]₆[KFI] | TATA | 51 |
| | (A-(SEQ ID NO: 74)-A[Sar]₆[KFI]) | | |
| BCY7375 | ACDPLCLSIKRQLGLCA[Sar]₆[KFI] | TATA | 66 |
| | (A-(SEQ ID NO: 75)-A[Sar]₆[KFI]) | | |
| BCY7376 | ACDPLCLSIKRKLGLCA[Sar]₆[KFI] | TATA | 73 |
| | (A-(SEQ ID NO: 76)-A[Sar]₆[KFI]) | | |
| BCY7377 | ACDPLCLSIKRGLGLCA[Sar]₆[KFI] | TATA | 114 |
| | (A-(SEQ ID NO: 77)-A[Sar]₆[KFI]) | | |
| BCY7384 | ACDMRCIRIKQSLGMCA[Sar]₆[KFI] | TATA | 932 |
| | (A-(SEQ ID NO: 78)-A[Sar]₆[KFI]) | | |
| BCY7385 | ACRDWCHWTFDNGHPCA[Sar]₆[KFI] | TATA | 58 |
| | (A-(SEQ ID NO: 79)-A[Sar]₆[KFI]) | | |
| BCY7386 | ACRDWCHWTFSHGTPCA[Sar]₆[KFI] | TATA | 879 |
| | (A-(SEQ ID NO: 80)-A[Sar]₆[KFI]) | | |
| BCY7387 | ACRDWCHWTFSHGHPCA[Sar]₆[KFI] | TATA | 1185 |
| | (A-(SEQ ID NO: 81)-A[Sar]₆[KFI]) | | |
| BCY7388 | ACRDWCHWTFTHGHPCA[Sar]₆[KFI] | TATA | 44 |
| | (A-(SEQ ID NO: 82)-A[Sar]₆[KFI]) | | |
| BCY7389 | ACRDWCHWTFTHSHPCA[Sar]₆[KFI] | TATA | 132 |
| | (A-(SEQ ID NO: 83)-A[Sar]₆[KFI]) | | |

### 2. PD-L1 Competition Binding Assay

Peptides without a fluorescent tag were tested in competition with FI-G-Sar5-ACPDPHNICHLWCA (Kd=68.75 nM—determined using the protocol above). Peptides were diluted to an appropriate concentration in assay buffer as described in the direct binding assay with a maximum of 5% DMSO, then serially diluted 1 in 2. Five μL of diluted peptide was added to the plate followed by 10 μL of human PD-L1, then 10 μL fluorescent peptide added. Measurements were conducted as for the direct binding assay, however the gain was determined prior to the first measurement. Data analysis was in Systat Sigmaplot version 12.0 where the mP values were fit to a user defined cubic equation to generate a Ki value:

$$f = y min + ( y max - y min ) / Lig * ( ( Lig * ( ( 2 * ( ( Klig + Kcomp + Lig + Comp - Prot * c ) \hat{} 2 - 3 * ( Kcomp * ( Lig - Prot * c ) + Klig * ( Comp - Prot * c ) + Klig * Kcomp ) ) \hat{} 0.5 * COS ( ARCCOS ( ( - 2 * ( Klig + Kcomp + Lig + Comp - Prot * c ) \hat{} 3 + 9 * ( Klig + Kcomp + Lig + Comp - Prot * c ) * ( Kcomp * ( Lig - Prot * c ) + Klig * ( Comp - Prot * c ) + Klig * Kcomp ) - 27 * ( - 1 * Klig * Kcomp * Prot * c ) ) \text{} / ( {2}^{*} ( ( ( ( Klig + Kcomp + Lig + Comp - Prot * c ) \hat{} 2 - 3 * ( Kcomp * ( Lig - Prot * c ) + Klig * ( Comp - Prot * c ) + Klig * Kcomp ) ) \hat{} 3 ) \hat{} 0.5 ) ) ) / 3 ) ) - ( Klig + Kcomp + Lig + Comp - Prot * c ) ) ) / ( ( 3 * Klig ) + ( ( 2 * ( ( Klig + Kcomp + Lig + Comp - Prot * c ) \hat{} 2 - 3 * ( Kcomp * ( Lig - Prot * c ) + Klig * ( Comp - Prot * c ) + Klig * Kcomp ) ) \hat{} 0.5 * COS ( ARCCOS ( ( - 2 * ( Klig + Kcomp + Lig + Comp - Prot * c ) \hat{} 3 + 9 * ( Klig + Kcomp + Lig + Comp - Prot * c ) * ( Kcomp * ( Lig - Prot * c ) + Klig * ( Comp - Prot * c ) + Klig * Kcomp ) - 27 * ( - 1 * Klig * Kcomp * Prot * c ) ) \text{} / ( 2 * ( ( ( ( Klig + Kcomp + Lig + Comp - Prot * c ) \hat{} 2 - 3 * ( Kcomp * ( Lig - Prot * c ) + Klig * ( Comp - Prot * c ) + Klig * Kcomp ) ) \hat{} 3 ) \hat{} 0.5 ) ) ) / 3 ) ) - ( Klig + Kcomp + Lig + Comp - Prot * c ) ) ) ) .$$

“Lig”, “KLig” and “Prot” were all defined values relating to: fluorescent peptide concentration, the Kd of the fluorescent peptide and PD-L1 concentration respectively.

The following selected bicyclic peptide ligands of the invention were tested in the above mentioned PD-L1 Competition Binding Assay and the results are shown in Table 3:

TABLE 3
Competition Binding Assay Data for Selected Bicyclic Peptide
Ligands of the Invention

| Peptide | | | |
|---|---|---|---|
| Number | Sequence | Scaffold | Ki (nM) |
| BCY519 | ACSWSWLTMCQKLHLCA | TATA | 29.6 ± 9.2 |
| | (A-(SEQ ID NO: 1)-A) | | |
| BCY521 | ACSTSWMNLCKQLNLCA | TATA | 170 |
| | (A-(SEQ ID NO: 2)-A) | | |
| BCY522 | ACSPIWTNTCIQLGLCA | TATA | 618 |
| | (A-(SEQ ID NO: 3)-A) | | |
| BCY523 | ACTPNWNAMCLKLNLCA | TATA | 362 |
| | (A-(SEQ ID NO: 4)-A) | | |
| BCY526 | ACDVFTHCILLAKPCA | TATA | 378.75 ± 96.42 |
| | (A-(SEQ ID NO: 5)-A) | | |
| BCY527 | ACSESWSNMCVSLGLCA | TATA | 439.5 ± 125.71 |
| | (A-(SEQ ID NO: 6)-A) | | |
| BCY528 | ACSAEWRNMCVQLDLCA | TATA | 112 ± 37.24 |
| | (A-(SEQ ID NO: 7)-A) | | |
| BCY529 | ACSASWSNMCVELGLCA | TATA | 201.3 ± 211.09 |
| | (A-(SEQ ID NO: 8)-A) | | |
| BCY530 | ACSDNWLNMCVELGLCA | TATA | 106.45 ± 89.28 |
| | (A-(SEQ ID NO: 9)-A) | | |
| BCY531 | ACSESWSAMCASLGLCA | TATA | 118.05 ± 86.14 |
| | (A-(SEQ ID NO: 10)-A) | | |
| BCY532 | ACSESWSNMCKSLGLCA | TATA | 243 |
| | (A-(SEQ ID NO: 11)-A) | | |
| BCY533 | ACSESWRNMCVQLNLCA | TATA | 19.2 ± 4.7 |
| | (A-(SEQ ID NO: 12)-A) | | |
| BCY534 | ACSPEWTNMCVQLHLCA | TATA | 65.75 ± 4.61 |
| | (A-(SEQ ID NO: 13)-A) | | |
| BCY535 | ACSTQWNNMCVQLGLCA | TATA | 115.65 ± 39.89 |
| | (A-(SEQ ID NO: 14)-A) | | |
| BCY537 | ACSSSWTNMCVQLGLCA | TATA | 49.45 ± 59.88 |
| | (A-(SEQ ID NO: 15)-A) | | |
| BCY538 | ACSAEWRNMCVELNLCA | TATA | 524 |
| | (A-(SEQ ID NO: 16)-A) | | |
| BCY539 | ACSPEWKNMCITLNLCA | TATA | 116 |
| | (A-(SEQ ID NO: 17)-A) | | |

### 3. PD-L1 SPR Binding Assay

Biacore experiments were performed to determine kₐ (M⁻¹s⁻¹), k_{d} (s⁻¹), K_{D} (nM) values of monomeric peptides binding to human PD-L1 protein. Recombinant human PD-L1 (Sino Biologicals or R&D systems) or mouse PD-L1 (R&D systems) was resuspended in PBS and biotinylated using EZ-Link™ Sulfo-NHS-LC-LC-Biotin reagent (Thermo Fisher) as per the manufacturer's suggested protocol. The protein was desalted to remove uncoupled biotin using spin columns into PBS. For analysis of binding, a Biacore 3000 instrument was used utilizing a CM5 sensor chip (GE Healthcare). Streptavidin was immobilized on the chip using standard amine-coupling chemistry at 25° C. with HBS-N(10 mM HEPES, 0.15 M NaCl, pH 7.4) as the running buffer. The carboxymethyl dextran surface was activated with a 12 min injection of a 1:1 ratio of 0.4 M 1-ethyl-3-(3-dimethylaminopropyl) carbodiimide hydrochloride (EDC)/0.1 M N-hydroxy succinimide (NHS) at a flow rate of 10 μl/min. For capture of streptavidin, protein was diluted to 0.1 mg/ml in 10 mM sodium acetate (pH 4.5) and captured by injecting 70 μl onto the activated chip surface. Residual activated groups were blocked with a 7 min injection of 1 M ethanolamine (pH 8.5). Biotinylated PD-L1 stock was diluted 1:100 in HBS-N and captured on one flow cell at 5 μl/min to a level of 1000-1300 RU. Buffer was changed to PBS/0.05% Tween 20 and a dilution series of the peptides was prepared in this buffer with a final DMSO concentration of 0.5%. The top concentrations were 200 nM or 500 nM with 6 2-fold dilutions. The SPR analysis was run at 25° C. at a flow rate of Flow rate 50 μl/min with 60 seconds association and 400 seconds disassociation. Data were corrected for DMSO excluded volume effects. All data were double-referenced for blank injections and reference surface using standard processing procedures and data processing and kinetic fitting were performed using Scrubber software, version 2.0c (Biologic Software). Data were fitted using mass transport model allowing for mass transport effects where appropriate.

The following selected bicyclic peptide ligands of the invention were tested in the above mentioned PD-L1 SPR Binding Assay and the results are shown in Tables 4 and 5:

TABLE 4
Human SPR Binding Assay Data for Selected Bicyclic Peptide
Ligands of the Invention

| Peptide | | | |
|---|---|---|---|
| Number | Sequence | Scaffold | Kd (nM) |
| BCY3835 | SDKCSAGWLTMCQKLHLCA | TATA | 30.1 |
| | (SDK-(SEQ ID NO: 19)-A) | | |
| BCY10051 | [Ac][dS]DKCSAGWLTMCQKLHLCA | TATA | 20.7 |
| | ([Ac]ID NO: 19)-A) | | |
| BCY10052 | SD[HArg]CSAGWLTMCQKLHLCA | TATA | 22.3 |
| | (SDID NO: 19)-A) | | |
| BCY10053 | SD[HSer]CSAGWLTMCQKLHLCA | TATA | 47.5 |
| | (SDID NO: 19)-A) | | |
| BCY10054 | SDKCS[Aib]GWLTMCQKLHLCA | TATA | 31.2 |
| | (SDK-(SEQ ID NO: 21)-A) | | |
| BCY10055 | SDKCS[Abu]GWLTMCQKLHLCA | TATA | 27.9 |
| | (SDK-(SEQ ID NO: 22)-A) | | |
| BCY10057 | SDKCSA[HSer]WLTMCQKLHLCA | TATA | 31.8 |
| | (SDK-(SEQ ID NO: 23)-A) | | |
| BCY10058 | SDKCSAGWLT[Nle]CQKLHLCA | TATA | 59.7 |
| | (SDK-(SEQ ID NO: 24)-A) | | |
| BCY10060 | SDKCS[dA]GWLTMCQKLHLCA | TATA | 229 |
| | (SDK-(SEQ ID NO: 25)-A) | | |
| BCY10061 | SDKCS[HSer]GWLTMCQKLHLCA | TATA | 33.7 |
| | (SDK-(SEQ ID NO: 26)-A) | | |
| BCY10062 | SDKCS[Nle]GWLTMCQKLHLCA | TATA | 23 |
| | (SDK-(SEQ ID NO: 27)-A) | | |
| BCY10064 | SDKCSAGWLT[HPhe]CQKLHLCA | TATA | 20.5 |
| | (SDK-(SEQ ID NO: 28)-A) | | |
| BCY10065 | SDKCSAGWLTMCVQLGLCA | TATA | 34.1 |
| | (SDK-(SEQ ID NO: 29)-A) | | |
| BCY10066 | SDKCSAGWLTMC[Chg]KLHLCA | TATA | 14.4 |
| | (SDK-(SEQ ID NO: 30)-A) | | |
| BCY10067 | SDKCSAGWLTMCLKLHLCA | TATA | 5.51 |
| | (SDK-(SEQ ID NO: 31)-A) | | |
| BCY10068 | SDKCSAGWLTMCQ[Nle]LHLCA | TATA | 54.5 |
| | (SDK-(SEQ ID NO: 32)-A) | | |
| BCY10069 | SDKCSAGWLTMCQ[HSer]LHLCA | TATA | 92 |
| | (SDK-(SEQ ID NO: 33)-A) | | |
| BCY10071 | SDKCSAGWLTMCQK[Nle]HLCA | TATA | 58.2 |
| | (SDK-(SEQ ID NO: 34)-A) | | |
| BCY10072 | SDKCSAGWLTMCQKL[dA]LCA | TATA | 22.5 |
| | (SDK-(SEQ ID NO: 35)-A) | | |
| BCY10073 | SDKCSAGWLTMCQKL[Aib]LCA | TATA | 15.8 |
| | (SDK-(SEQ ID NO: 36)-A) | | |
| BCY10074 | SDKCSAGWLTMCQKLH[Nle]CA | TATA | 36.1 |
| | (SDK-(SEQ ID NO: 37)-A) | | |
| BCY10075 | [Ac]SDKCSAGWLTMCQKLHLCA | TATA | 28.6 |
| | ([Ac]SDK-(SEQ ID NO: 19)-A) | | |
| BCY10076 | [Ac]SDRCSAGWLTMCQKLHLC | TATA | 14.5 |
| | ([Ac]SDR-(SEQ ID NO: 19)) | | |
| BCY10078 | [Ac]SDKCSAGWLTMCQRLHLC | TATA | 12.2 |
| | ([Ac]SDK-(SEQ ID NO: 38)) | | |
| BCY10083 | SDKCSAGWLTMCQKLH[Nva]CA | TATA | 75 |
| | (SDK-(SEQ ID NO: 39)-A) | | |
| BCY10084 | SDKCSAGWLTMCQK[Nva]HLCA | TATA | 43.2 |
| | (SDK-(SEQ ID NO: 40)-A) | | |
| BCY10085 | SDKCSAGWLT[Nva]CQKLHLCA | TATA | 32.5 |
| | (SDK-(SEQ ID NO: 41)-A) | | |
| BCY10467 | [Ac]SDKCSAGWLTMCKQLNLCPSH | TATA | 3.1 |
| | ([Ac]SDK-(SEQ ID NO: 42)-PSH) | | |
| BCY10934 | [Ac]SDKCSAGWLTMCKQLNLOPS | TATA | 10.9 |
| | ([Ac]SDK-(SEQ ID NO: 42)-PS) | | |
| BCY10935 | [Ac]SDKCSAGWLTMCKQLNLOP | TATA | 14.9 |
| | ([Ac]SDK-(SEQ ID NO: 42)-P) | | |
| BCY10936 | [Ac]SDKCSAGWLTMCKQLNLOPS-COOH | TATA | 13 |
| | ([Ac]SDK-(SEQ ID NO: 42)-PS-COOH) | | |
| BCY10937 | [Ac]SDKCSAGWLTMCKQLNLOP-COOH | TATA | 5.03 |
| | ([Ac]SDK-(SEQ ID NO: 42)-P-COOH) | | |
| BCY10938 | [Ac]SDKCSAGWLTMCKQLNLC-COOH | TATA | 12.2 |
| | ([Ac]SDK-(SEQ ID NO: 42)-COOH) | | |
| BCY10939 | [Ac]SDKCSAGWLTMCKQLNLCPSH-COOH | TATA | 3.39 |
| | ([Ac]SDK-(SEQ ID NO: 42)-PSH-COOH) | | |
| BCY10940 | [Ac]DKCSAGWLTMCKQLNLCPSH | TATA | 2.32 |
| | (+AcID NO: 42)-PSH) | | |
| BCY10941 | [Ac]KCSAGWLTMCKQLNLCPSH | TATA | 3.67 |
| | ([Ac]K-(SEQ ID NO: 42)-PSH) | | |
| BCY10942 | [Ac]CSAGWLTMCKQLNLCPSH | TATA | 24.1 |
| | ([Ac]-(SEQ ID NO: 42)-PSH) | | |
| BCY10943 | [Ac]SDKCSAGWLTMCQKLHLCPSH | TATA | 7.76 |
| | ([Ac]SDK-(SEQ ID NO: 19)-PSH) | | |
| BCY10944 | [Ac]SD[HArg]CS[Aib]GWLT[hPhe]CLKL[Aib]LCPSH | TATA | 10.7 |
| | ([Ac]SDID NO: 43)-PSH) | | |
| BCY10945 | [Ac]SDKCSAG[7-AzaW]LTMCQKLHLC | TATA | 147 |
| | ([Ac]SDK-(SEQ ID NO: 44)) | | |
| BCY10946 | [Ac]SDKCSAGWLTMC[Aad]KLHLC | TATA | 13.1 |
| | ([Ac]SDK-(SEQ ID NO: 45)) | | |
| BCY10947 | [Ac]SDKCSAEWLTMCQKLHLC | TATA | 31.1 |
| | ([Ac]SDK-(SEQ ID NO: 46)) | | |
| BCY10948 | [Ac]SDKCSA[Aad]WLTMCQKLHLC | TATA | 97.5 |
| | ([Ac]SDK-(SEQ ID NO: 47)) | | |
| BCY10949 | [Ac]SDKCSAGWLTMCEKLHLC | TATA | 18.7 |
| | ([Ac]SDK-(SEQ ID NO: 48)) | | |
| BCY10950 | [Ac]SDKCSAGWLTMC[Cpa]KLHLC | TATA | 8.73 |
| | ([Ac]SDK-(SEQ ID NO: 49)) | | |
| BCY10951 | [Ac]SEKCSAGWLTMCQKLHLC | TATA | 7.28 |
| | ([Ac]SEK-(SEQ ID NO: 19)) | | |
| BCY10952 | [Ac]SDKC[AlloThr]AGWLTMCQKLHLC | TATA | 108 |
| | ([Ac]SDK-(SEQ ID NO: 50)) | | |
| BCY10954 | [Ac]SDKCSAGW+[Cpa]TMCQKLHLC | TATA | 19.4 |
| | ([Ac]SDK-(SEQ ID NO: 51)) | | |
| BCY10956 | [Ac]SDKCSAGWLTMCQKLH[Cpa]C | TATA | 24.9 |
| | ([Ac]SDK-(SEQ ID NO: 52)) | | |
| BCY10959 | [Ac]SD[HArg]CS[Nle]GWLT[hPhe]CLKL[Aib]LCPSH | TATA | 5.54 |
| | ([Ac]SDID NO: 53)-PSH) | | |
| BCY10960 | [Ac]SDKCSEGWLTMCQKLHLC | TATA | 9.94 |
| | ([Ac]SDK-(SEQ ID NO: 54)) | | |
| BCY10961 | [Ac]SDKCS[Aad]GWLTMCQKLHLC | TATA | 7.42 |
| | ([Ac]SDK-(SEQ ID NO: 55)) | | |
| BCY519 | ACSWSWLTMCQKLHLCA | TATA | 60.9 |
| | (A-(SEQ ID NO: 1)-A) | | |
| BCY527 | ACSESWSNMCVSLGLCA | TATA | 933 |
| | (A-(SEQ ID NO: 6)-A) | | |
| BCY528 | ACSAEWRNMCVQLDLCA | TATA | 291 |
| | (A-(SEQ ID NO: 7)-A) | | |
| BCY529 | ACSASWSNMCVELGLCA | TATA | 442 |
| | (A-(SEQ ID NO: 8)-A) | | |
| BCY11830 | Ac-SDKCSAGWLTMCLKLHLCPSH | TATA | 24.1 |
| | ([Ac]-SDK-(SEQ ID NO: 31)-PSH) | | |
| BCY11831 | Ac-SEKCS[Aad]GWLTMCLKLHLCPSH | TATA | 13.2 |
| | (Ac-SEK-(SEQ ID NO: 85)-PSH) | | |
| BCY11833 | Ac-ACSAGWLTMCQKLHLCPSH | TATA | 32.5 |
| | ([Ac]-A-(SEQ ID NO: 19)-PSH) | | |
| BCY11853 | Ac-DKCS[Aad]GWLTMCQKLHLCPSH-COOH | TATA | 17.6 |
| | (Ac-DK-(SEQ ID NO: 84)-PSH-COOH) | | |
| BCY11818 | [Ac]DKCS[Aad]GWLTMCQKLHLC | TATA | 30.1 |
| | (Ac-DK-(SEQ ID NO: 84)) | | |
| BCY11819 | [Ac]KCS[Aad]GWLTMCQKLHLC | TATA | 27.3 |
| | (Ac-K-(SEQ ID NO: 84)) | | |
| BCY11820 | [Ac]SEKCS[Aad]GWLTMCQKLHLC | TATA | 17.4 |
| | (Ac-SEK-(SEQ ID NO: 84)) | | |
| BCY11821 | [Ac]EKCS[Aad]GWLTMCQKLHLC | TATA | 17.5 |
| | (Ac-EK-(SEQ ID NO: 84)) | | |
| BCY11822 | [Ac]SDKCS[Aad]GWLTMCLKLHLC | TATA | 16.5 |
| | (Ac-SDK-(SEQ ID NO: 85)) | | |
| BCY11823 | [Ac]DKCS[Aad]GWLTMCLKLHLC | TATA | 17.7 |
| | (Ac-DK-(SEQ ID NO: 85)) | | |
| BCY11824 | Ac-KCS[Aad]GWLTMCLKLHLC | TATA | 60.0 |
| | (Ac-K-(SEQ ID NO: 85)) | | |
| BCY11825 | Ac-SEKCS[Aad]GWLTMCLKLHLC | TATA | 28.8 |
| | (Ac-SEK-(SEQ ID NO: 85)) | | |
| BCY11826 | Ac-EKCS[Aad]GWLTMCLKLHLC | TATA | 18.2 |
| | (Ac-EK-(SEQ ID NO: 85)) | | |
| BCY11827 | Ac-[HArg]CS[Aad]GWLTMCLKLHLC | TATA | 30.0 |
| | (Ac-ID NO: 85)) | | |
| BCY11828 | Ac-SDKCS[Aad]GWLTMCLKLHLCA | TATA | 16.6 |
| | (Ac-SDK-(SEQ ID NO: 85)-A) | | |
| BCY11829 | Ac-SDKCS[Aad]GWL[3HyV]MCLKLHLC | TATA | 43.6 |
| | (Ac-SDK-(SEQ ID NO: 86)) | | |
| BCY11832 | Ac-SDKCSAGWLTMCQKLHLC | TATA | 68.4 |
| | ([Ac]-SDK-(SEQ ID NO: 19)) | | |

NB: No Binding at 500 nM

TABLE 5
Mouse SPR Binding Assay Data for Selected Bicyclic
Peptide Ligands of the Invention

| Peptide | | | Kd |
|---|---|---|---|
| Number | Sequence | Scaffold | (nM) |
| BCY8938 | [PYA][B-Ala][Sar]₁₀C[HArg]DWCHWTFSHGHPC | TATA | 117 |
| | ([PYA][B-Ala]-[Sar₁₀]-(SEQ ID NO: 18)) | | |

### 3. PD-1/PD-L1 Blockade Bioassay

Effector Jurkat cells engineered to overexpress human PD-1 and express a luciferase gene under the NFAT response element were purchased from Promega. Target CHO-K1 cells which overexpress human PD-L1 and an engineered cell surface protein designed to activate the Jurkat's T cell receptor in an antigen-independent manner were also purchased from Promega. CHO-K1 cells were plated the day before the assay in Ham's F-12 Medium+10% FBS and were allowed to adhere to the plate. On the day of the assay, media was removed and Jurkat cells were plated with a dose response of test articles in RPM11640 media with 1% FBS (80 μL total volume per well). A PD-L1 monoclonal antibody (PD-L1 mAb) was included as a positive control (clone MIH1, Invitrogen catalog #16598382). After 6 hours of incubation at 37° C., 5% CO₂, 80 μL of Bio-Glo reagent (Promega) was added to each well and allowed to equilibrate for 10 minutes at room temperature. Luminescence was read on the Clariostar plate reader (BMG LabTech). Fold induction was calculated by dividing the luminescence signal by the average of the background wells (wells with both cell lines and no test article). Data was graphed in Prism and fit to a 4-parameter logistic curve.

The results are shown in FIG. 1 and Table 6 where it can be seen that the PD-L1 binding bicyclic peptides (BCY10467, BCY10939 and BCY10959) demonstrated the ability to block PD-1/PD-L1 interaction between PD-1 expressing T cells and CHO-K1 stable expressing PD-L1.

TABLE 6
PD-1/PD-L1 Blockade Bioassay Results

| Compound | EC50 (nM) |
|---|---|
| BCY10467 | 195.5 |
| BCY10939 | 211.0 |
| BCY10959 | 119.5 |
| PD-L1 mAb | 2.458 |

1. A peptide ligand specific for PD-L1 comprising a polypeptide comprising at least three reactive groups, separated by at least two loop sequences, and a non-aromatic molecular scaffold which forms covalent bonds with the reactive groups of the polypeptide such that at least two polypeptide loops are formed on the molecular scaffold.

2-13. (canceled)

14. A method for preventing, suppressing or treating a disease or disorder mediated by PD-L1, comprising: administering to a patient in need thereof
(a) peptide ligand specific for PD-L1, or
(b) a drug conjugate comprising a peptide ligand specific for PD-L1 conjugated to one or more effector and/or functional groups,
wherein the peptide ligand comprises (i) a polypeptide comprising at least three reactive groups separated by at least two loop sequences and (ii) a non-aromatic molecular scaffold that forms covalent bonds with the reactive groups of the polypeptide such that at least two polypeptide loops are formed on the molecular scaffold.

15. The method of claim 14, wherein each of the at least two loop sequences comprises 3 amino acids.

16. The method of claim 14, wherein the reactive groups are selected from cysteine and penicillamine residues.

17. The method of claim 14, wherein the peptide ligand comprises an amino acid sequence selected from:
(SEQ ID NO: 1)
CᵢSWSWLTMCᵢᵢQKLHLCᵢᵢᵢ;
(SEQ ID NO: 2)
CᵢSTSWMNLCᵢᵢKQLNLCᵢᵢᵢ;
(SEQ ID NO: 3)
CᵢSPIWTNTCᵢᵢIQLGLCᵢᵢᵢ;
(SEQ ID NO: 4)
CᵢTPNWNAMCᵢᵢLKLNLCᵢᵢᵢ;
(SEQ ID NO: 5)
CᵢDVFTHCᵢᵢILLAKPCᵢᵢᵢ;
(SEQ ID NO: 6)
CᵢSESWSNMCᵢᵢVSLGLCᵢᵢᵢ;
(SEQ ID NO: 7)
CᵢSAEWRNMCᵢᵢVQLDLCᵢᵢᵢ;
(SEQ ID NO: 8)
CᵢSASWSNMCᵢᵢVELGLCᵢᵢᵢ;
(SEQ ID NO: 9)
CᵢSDNWLNMCᵢᵢVELGLCᵢᵢᵢ;
(SEQ ID NO: 10)
CᵢSESWSAMCᵢᵢASLGLCᵢᵢᵢ;
(SEQ ID NO: 11)
CᵢSESWSNMCᵢᵢKSLGLCᵢᵢᵢ;
(SEQ ID NO: 12)
CᵢSESWRNMCᵢᵢVQLNLCᵢᵢᵢ;
(SEQ ID NO: 13)
CᵢSPEWTNMCᵢᵢVQLHLCᵢᵢᵢ;
(SEQ ID NO: 14)
CᵢSTQWNNMCᵢᵢVQLGLCᵢᵢᵢ;
(SEQ ID NO: 18)
CᵢSSSWTNMCᵢᵢVQLGLCᵢᵢᵢ;
(SEQ ID NO: 16)
CᵢSAEWRNMCᵢᵢVELNLCᵢᵢᵢ;
(SEQ ID NO: 17)
CᵢSPEWKNMCᵢᵢITLNLCᵢᵢᵢ;
(SEQ ID NO: 18)
Cᵢ[HArg]DWCᵢᵢHWTFSHGHPCᵢᵢᵢ;
(SEQ ID NO: 19)
CᵢSAGWLTMCᵢᵢQKLHLCᵢᵢᵢ;
(SEQ ID NO: 20)
CᵢSAGWLTMCᵢᵢQ[K(PYA)]LHLCᵢᵢᵢ;
(SEQ ID NO: 21)
CᵢS[Aib]GWLTMCᵢᵢQKLHLCᵢᵢᵢ;
(SEQ ID NO: 22)
CᵢS[Abu]GWLTMCᵢᵢQKLHLCᵢᵢᵢ;
(SEQ ID NO: 23)
CᵢSA[HSer]NLTMCᵢᵢQKLHLCᵢᵢᵢ;
(SEQ ID NO: 24)
CᵢSAGWLT[Nle]CᵢᵢQKLHLCᵢᵢᵢ;
(SEQ ID NO: 25)
CᵢS[dA]GWLTMCᵢᵢQKLHLCᵢᵢᵢ;
(SEQ ID NO: 26)
CᵢS[HSer]GWLTMCᵢᵢQKLHLCᵢᵢᵢ;
(SEQ ID NO: 27)
CᵢS[Nle]GWLTMCᵢᵢQKLHLCᵢᵢᵢ;
(SEQ ID NO: 28)
CᵢSAGWLT[HPhe]CᵢᵢQKLHLCᵢᵢᵢ;
(SEQ ID NO: 29)
CᵢSAGWLTMCᵢᵢVQLGLCᵢᵢᵢ;
(SEQ ID NO: 30)
CᵢSAGWLTMCᵢᵢ[Chg]KLHLCᵢᵢᵢ;
(SEQ ID NO: 31)
CᵢSAGWLTMCᵢᵢLKLHLCᵢᵢᵢ;
(SEQ ID NO: 32)
CᵢSAGWLTMCᵢᵢQ[Nle]LHLCᵢᵢᵢ;
(SEQ ID NO: 33)
CᵢSAGWLTMCᵢᵢQ[HSer]LHLCᵢᵢᵢ;
(SEQ ID NO: 34)
CᵢSAGWLTMCᵢᵢQK[Nle]HLCᵢᵢᵢ;
(SEQ ID NO: 35)
CᵢSAGWLTMCᵢᵢQKL[dA]LCᵢᵢᵢ;
(SEQ ID NO: 36)
CᵢSAGWLTMCᵢᵢQKL[Aib]LCᵢᵢᵢ;
(SEQ ID NO: 37)
CᵢSAGWLTMCᵢᵢQKLH[Nle]Cᵢᵢᵢ;
(SEQ ID NO: 38)
CᵢSAGWLTMCᵢᵢQRLHLCᵢᵢᵢ;
(SEQ ID NO: 39)
CᵢSAGWLTMCᵢᵢQKLH[Nva]Cᵢᵢᵢ;
(SEQ ID NO: 40)
CᵢSAGWLTMCᵢᵢQK[Nva]HLCᵢᵢᵢ;
(SEQ ID NO: 41)
CᵢSAGWLT[Nva]CᵢᵢQKLHLCᵢᵢᵢ;
(SEQ ID NO: 42)
CᵢSAGWLTMCᵢᵢHKQLNLCᵢᵢᵢ;
(SEQ ID NO: 43)
CᵢS[Aib]GWLT[HPhe]CᵢᵢLKL[Aib]LCᵢᵢᵢ;
(SEQ ID NO: 44)
CᵢSAG[7-AzaW]LTMCᵢᵢQKLHLCᵢᵢᵢ;
(SEQ ID NO: 45)
CᵢSAGWLTMCᵢᵢ[Aad]KLHLCᵢᵢᵢ;
(SEQ ID NO: 46)
CᵢSAEWLTMCᵢᵢQKLHLCᵢᵢᵢ;
(SEQ ID NO: 47)
CᵢSA[Aad]WLTMCᵢᵢQKLHLCᵢᵢᵢ;
(SEQ ID NO: 48)
CᵢSAGWLTMCᵢᵢEKLHLCᵢᵢᵢ;
(SEQ ID NO: 49)
CᵢSAGWLTMCᵢᵢ[Cpap]KLHLCᵢᵢᵢ;
(SEQ ID NO: 50)
Cᵢ[AlloThr]AGWLTMCᵢᵢQKLHLCᵢᵢᵢ;
(SEQ ID NO: 51)
CᵢSAGW[Cpa]TMCᵢᵢQKLHLCᵢᵢᵢ;
(SEQ ID NO: 52)
CᵢSAGWLTMCᵢᵢQKLH[Cpa]Cᵢᵢᵢ;
(SEQ ID NO: 53)
CᵢS[Nle]GWLT[HPhe]CᵢᵢLKL[Aib]LCᵢᵢᵢ;
(SEQ ID NO: 54)
CᵢSEGWLTMCᵢᵢQKLHLCᵢᵢᵢ;
(SEQ ID NO: 55)
CᵢS[Aad]GWLTMCᵢᵢQKLHLCᵢᵢᵢ;
(SEQ ID NO: 56)
CᵢSDQWMQMCᵢᵢSKLTCᵢᵢᵢ;
(SEQ ID NO: 57)
CᵢSDGWLTMCᵢᵢQKLHLCᵢᵢᵢ;
(SEQ ID NO: 58)
CᵢSDEWLTMCᵢᵢQKLHLCᵢᵢᵢ;
(SEQ ID NO: 59)
CᵢSNSWLTMCᵢᵢQKLHLCᵢᵢᵢ;
(SEQ ID NO: 60)
CᵢSPAWLTMCᵢᵢQKLHLCᵢᵢᵢ;
(SEQ ID NO: 61)
CᵢSPEWLTMCᵢᵢQKLHLCᵢᵢᵢ;
(SEQ ID NO: 62)
CᵢSPGWLTMCᵢᵢQKLHLCᵢᵢᵢ;
(SEQ ID NO: 63)
CᵢSPQWLTMCᵢᵢQKLHLCᵢᵢᵢ;
(SEQ ID NO: 64)
CᵢSPSWLTMCᵢᵢQKLHLCᵢᵢᵢ;
(SEQ ID NO: 65)
CᵢSDSWKTMCᵢᵢQKLHLCᵢᵢᵢ;
(SEQ ID NO: 66)
CᵢSESWSTMCᵢᵢQKLHLCᵢᵢᵢ;
(SEQ ID NO: 67)
CᵢSPSWRTMCᵢᵢQKLHLCᵢᵢᵢ;
(SEQ ID NO: 68)
CᵢSPSWRNMCᵢᵢQKLHLCᵢᵢᵢ;
(SEQ ID NO: 69)
CᵢSWSWLTMCᵢᵢKQLNLCᵢᵢᵢ;
(SEQ ID NO: 70)
CᵢSWSWLTMCᵢᵢQKLDLCᵢᵢᵢ;
(SEQ ID NO: 71)
CᵢSSSWLTMCᵢᵢQKLHLCᵢᵢᵢ;
(SEQ ID NO: 72)
CᵢSWSWLNMCᵢᵢQKLHLCᵢᵢᵢ;
(SEQ ID NO: 73)
CᵢDPLCᵢᵢLSIRASLGLCᵢᵢᵢ;
(SEQ ID NO: 74)
CᵢDPLCᵢᵢLSIKRSLGLCᵢᵢᵢ;
(SEQ ID NO: 75)
CᵢDPLCᵢᵢLSIKRQLGLCᵢᵢᵢ;
(SEQ ID NO: 76)
CᵢDPLCᵢᵢLSIKRKLGLCᵢᵢᵢ;
(SEQ ID NO: 77)
CᵢDPLCᵢᵢLSIKRGLGLCᵢᵢᵢ;
(SEQ ID NO: 78)
CᵢDMRCᵢᵢIRIKQSLGMCᵢᵢᵢ;
(SEQ ID NO: 79)
CᵢRDWCᵢᵢHWTFDNGHPCᵢᵢᵢ;
(SEQ ID NO: 80)
CᵢRDWCᵢᵢHWTFSHGTPCᵢᵢᵢ;
(SEQ ID NO: 81)
CᵢRDWCᵢᵢHWTFSHGHPCᵢᵢᵢ;
(SEQ ID NO: 82)
CᵢRDWCᵢᵢHWTFTHGHPCᵢᵢᵢ;
(SEQ ID NO: 83)
CᵢRDWCᵢᵢHWTFTHSHPCᵢᵢᵢ;
(SEQ ID NO: 84)
CᵢS[Aad]GWLTMCᵢᵢQKLHLCᵢᵢᵢ;
(SEQ ID NO: 85)
CᵢS[Aad]GWLTMCᵢᵢLKLHLCᵢᵢᵢ;
(SEQ ID NO: 86)
CᵢS[Aad]GWL[3HyV]MCᵢᵢLKLHLCᵢᵢᵢ;
(SEQ ID NO: 87)
CᵢSKGWLTMCᵢᵢQ[K(Ac)]LHCᵢᵢᵢ;
(SEQ ID NO: 88)
CᵢSAGWLTKCᵢᵢQ[K(Ac)]LHLCᵢᵢᵢ;
(SEQ ID NO: 89)
CᵢSAGWLTMCᵢᵢK[K(Ac)]LHLCᵢᵢᵢ;
(SEQ ID NO: 90)
CᵢSAGWLTMCᵢᵢQ[K(Ac)]LKLCᵢᵢᵢ;
(SEQ ID NO: 91)
CᵢSAGWLTMCᵢᵢQ[HArg]LHLCᵢᵢᵢ;
(SEQ ID NO: 92)
CᵢSAGWLTMCᵢᵢ[HArg]QLNLCᵢᵢᵢ;
(SEQ ID NO: 93)
CᵢS[Aad]GWLTMCᵢᵢKQLNLCᵢᵢᵢ;
(SEQ ID NO: 94)
CᵢS[Aad]G[1Nal]LTMCᵢᵢKQLNLCᵢᵢᵢ;
(SEQ ID NO: 95)
[Pen]ᵢS[Aad]GWLTMCᵢᵢKQLNLCᵢᵢᵢ;
(SEQ ID NO: 96)
CᵢS[Aad]GWLTM[Pen]ᵢᵢKQLNLCᵢᵢᵢ;
and
(SEQ ID NO: 97)
CᵢS[Aad]GWLTMCᵢᵢKQLNL[Pen]ᵢᵢᵢ;
wherein Cᵢ, [Pen]ᵢ, Cᵢᵢ [Pen]ᵢᵢ, Cᵢᵢᵢ and [Pen]ᵢᵢᵢ represent first, second and third reactive groups, respectively, HArg represents homoarginine, HSer represents homoserine, HPhe represents homophenylalanine, Aib represents 2-aminoisobutyric acid, Abu represents 2-aminobutyric acid, 2Nal represents 3-(2-Naphthyl)-L-alanine, Chg represents Cyclohexylglycine, Nva represents Norvaline, 7-AzaW represents 7-azatryptophan, Aad represents 2-aminoadipic acid, Cpa represents O-cyclopropylalanine, Dab represents 2,4-diaminobutyric acid, 3HyV represents 2-amino-3-hydroxy-3-methyl-butyric acid, Nle represents norleucine, Pen represents penicillamine and PYA represents 4-pentynoic acid, or a pharmaceutically acceptable salt thereof.

18. The method of claim 17, wherein the peptide ligand comprises an amino acid sequence selected from:
(B-Ala)-Sar5-A-(SEQ ID NO: 1)-A;
A-(SEQ ID NO: 1)-A;
A-(SEQ ID NO: 2)-A;
A-(SEQ ID NO: 3)-A;
A-(SEQ ID NO: 4)-A;
A-(SEQ ID NO: 5)-A;
A-(SEQ ID NO: 6)-A;
A-(SEQ ID NO: 7)-A;
A-(SEQ ID NO: 8)-A;
A-(SEQ ID NO: 9)-A;
A-(SEQ ID NO: 10)-A;
A-(SEQ ID NO: 11)-A;
A-(SEQ ID NO: 12)-A;
A-(SEQ ID NO: 13)-A;
A-(SEQ ID NO: 14)-A;
A-(SEQ ID NO: 15)-A;
A-(SEQ ID NO: 16)-A;
A-(SEQ ID NO: 17)-A;
[PYA]-[B-Ala]-[Sar₁₀]-(SEQ ID NO: 18);
SDK-(SEQ ID NO: 19)-A;
[PYA]-[B-Ala]-[Sar₁₀]-SDK-(SEQ ID NO: 19);
NH₂-SDK-(SEQ ID NO: 19)-[Sar₁₀]-[K(PYA)];
[Ac][dS]DK-(SEQ ID NO: 19)-A;
SD[HArg]-(SEQ ID NO: 19)-A;
SD[HSer]-(SEQ ID NO: 19)-A;
[Ac]SDK-(SEQ ID NO: 19)-A;
[Ac]SDR-(SEQ ID NO: 19);
[Ac]SDK-(SEQ ID NO: 19)-PSH;
[Ac]SEK-(SEQ ID NO: 19);
[Ac]-SDK-(SEQ ID NO: 19);
[Ac]-A-(SEQ ID NO: 19)-PSH;
SDK-(SEQ ID NO: 19)-A[Sar]₆;
[Ac]D[HArg]-(SEQ ID NO: 19);
NH₂-SDK-(SEQ ID NO: 20);
Ac-SDK-(SEQ ID NO: 20)-PSH;
Ac-SDK-(SEQ ID NO: 20);
SDK-(SEQ ID NO: 21)-A;
SDK-(SEQ ID NO: 22)-A;
SDK-(SEQ ID NO: 23)-A;
SDK-(SEQ ID NO: 24)-A;
SDK-(SEQ ID NO: 25)-A;
SDK-(SEQ ID NO: 26)-A;
SDK-(SEQ ID NO: 27)-A;
SDK-(SEQ ID NO: 28)-A;
SDK-(SEQ ID NO: 29)-A;
SDK-(SEQ ID NO: 30)-A;
SDK-(SEQ ID NO: 31)-A;
[Ac]-SDK-(SEQ ID NO: 31)-PSH;
SDK-(SEQ ID NO: 32)-A;
SDK-(SEQ ID NO: 33)-A;
SDK-(SEQ ID NO: 34)-A;
SDK-(SEQ ID NO: 35)-A;
SDK-(SEQ ID NO: 36)-A;
SDK-(SEQ ID NO: 37)-A;
[Ac]SDK-(SEQ ID NO: 38);
SDK-(SEQ ID NO: 39)-A;
SDK-(SEQ ID NO: 40)-A;
SDK-(SEQ ID NO: 41)-A;
[Ac]SDK-(SEQ ID NO: 42)-PSH;
[Ac]SDK-(SEQ ID NO: 42)-PS;
[Ac]SDK-(SEQ ID NO: 42)-P;
[Ac]SDK-(SEQ ID NO: 42)-PS-COOH;
[Ac]SDK-(SEQ ID NO: 42)-P-COOH;
[Ac]SDK-(SEQ ID NO: 42)-COOH;
[Ac]SDK-(SEQ ID NO: 42)-PSH-COOH;
[Ac]DK-(SEQ ID NO: 42)-PSH;
[Ac]K-(SEQ ID NO: 42)-PSH;
[Ac]-(SEQ ID NO: 42)-PSH;
[Ac]SD[HArg]-(SEQ ID NO: 43)-PSH;
[Ac]SDK-(SEQ ID NO: 44);
[Ac]SDK-(SEQ ID NO: 45);
[Ac]SDK-(SEQ ID NO: 46);
[Ac]SDK-(SEQ ID NO: 47);
[Ac]SDK-(SEQ ID NO: 48);
[Ac]SDK-(SEQ ID NO: 49);
[Ac]SDK-(SEQ ID NO: 50);
[Ac]SDK-(SEQ ID NO: 51);
[Ac]SDK-(SEQ ID NO: 52);
[Ac]SD[HArg]-(SEQ ID NO: 53)-PSH;
[Ac]SDK-(SEQ ID NO: 54);
[Ac]SDK-(SEQ ID NO: 55);
[B-Ala] [Sar]5A-(SEQ ID NO: 56)-A;
VER-(SEQ ID NO: 57)-A[Sar]₆;
REN-(SEQ ID NO: 58)-A[Sar]₆;
QQE-(SEQ ID NO: 59)-A[Sar]₆;
SGK-(SEQ ID NO: 59)-A[Sar]₆;
AGS-(SEQ ID NO: 60)-A[Sar]₆;
AQT-(SEQ ID NO: 61)-A[Sar]₆;
APV-(SEQ ID NO: 62)-A[Sar]₆;
ADV-(SEQ ID NO: 63)-A[Sar]₆;
GNK-(SEQ ID NO: 64)-A[Sar]₆;
KPK-(SEQ ID NO: 64)-A[Sar]₆;
ERV-(SEQ ID NO: 65)-A[Sar]₆;
AER-(SEQ ID NO: 66)-A[Sar]₆;
KEL-(SEQ ID NO: 67)-A[Sar]₆;
G[Sar]5KEL-(SEQ ID NO: 67)-A;
KEL-(SEQ ID NO: 68)-A[Sar]₆;
A-(SEQ ID NO: 69)-PSH[Sar]₆;
A-(SEQ ID NO: 70)-DHEN[Sar]₆;
REE-(SEQ ID NO: 71)-A[Sar]₆;
QAEK-(SEQ ID NO: 72)-A[Sar]₆;
A-(SEQ ID NO: 73)-A[Sar]₆;
A-(SEQ ID NO: 74)-A[Sar]₆;
A-(SEQ ID NO: 75)-A[Sar]₆;
A-(SEQ ID NO: 76)-A[Sar]₆;
A-(SEQ ID NO: 77)-A[Sar]₆;
A-(SEQ ID NO: 78)-A[Sar]₆;
A-(SEQ ID NO: 79)-A[Sar]₆;
A-(SEQ ID NO: 80)-A[Sar]₆;
A-(SEQ ID NO: 81)-A[Sar]₆;
A-(SEQ ID NO: 82)-A[Sar]₆;
A-(SEQ ID NO: 83)-A[Sar]₆;
Ac-DK-(SEQ ID NO: 84);
Ac-K-(SEQ ID NO: 84);
Ac-SEK-(SEQ ID NO: 84);
Ac-EK-(SEQ ID NO: 84);
Ac-DK-(SEQ ID NO: 84)-PSH-COOH;
Ac-SDK-(SEQ ID NO: 85);
Ac-DK-(SEQ ID NO: 85);
Ac-K-(SEQ ID NO: 85);
Ac-SEK-(SEQ ID NO: 85);
Ac-EK-(SEQ ID NO: 85);
Ac-[HArg]-(SEQ ID NO: 85);
Ac-SDK-(SEQ ID NO: 85)-A;
Ac-SEK-(SEQ ID NO: 85)-PSH;
Ac-SDK-(SEQ ID NO: 86);
Ac-D[HArg]-(SEQ ID NO: 87)-PSH;
Ac-D[HArg]-(SEQ ID NO: 88)-PSH;
Ac-D[HArg]-(SEQ ID NO: 89)-PSH;
Ac-D[HArg]-(SEQ ID NO: 90)-PSH;
Ac-SD[HArg]-(SEQ ID NO: 91)-PSHK;
Ac-SD[HArg]-(SEQ ID NO: 92)-PSHK; Ac-[HArg]-(SEQ
ID NO: 93)-PSH;
Ac-[HArg]-(SEQ ID NO: 94)-PSH;
Ac-[HArg]-(SEQ ID NO: 95)-PSH;
Ac-[HArg]-(SEQ ID NO: 96)-PSH;
and
Ac-[HArg]-(SEQ ID NO: 97)-PSH;
wherein PYA represents 4-pentynoic acid, B-Ala represents beta-alanine, Sar represents sarcosine, HSer represents homoserine and HArg represents homoarginine, or a pharmaceutically acceptable salt thereof.

19. The method of claim 14, wherein the molecular scaffold comprises 1,1′,1″-(1,3,5-triazinane-1,3,5-triyl)triprop-2-en-1-one (TATA).

20. The method of claim 17, wherein the pharmaceutically acceptable salt is selected from a free acid or a sodium, potassium, calcium, or ammonium salt.

21. The method of claim 18, wherein the pharmaceutically acceptable salt is selected from a free acid or a sodium, potassium, calcium, or ammonium salt.

22. The method of claim 14, wherein the PD-L1 is a human PD-L1.

23. The method of claim 14, wherein the PD-L1 is a mouse PD-L1.

24. The method of claim 14, wherein the drug conjugate is administered to the patient intravenously or intramuscularly.

25. The method of claim 14, wherein the disease or disorder mediated by PD-L1 is a cancer.

26. The method of claim 25, wherein the cancer is selected from kidney cancer, ovary cancer, bladder cancer, breast cancer, gastric cancer, and pancreatic cancer.

27. The method of claim 25, wherein the cancer is a liver cancer.

28. The method of claim 25, wherein the cancer is a hematopoietic malignancy.

29. The method of claim 28, wherein the hematopoietic malignancy is selected from non-Hodgkin's lymphoma (NHL), Burkitt's lymphoma (BL), multiple myeloma (MM), B chronic lymphocytic leukemia (B-CLL), B and T acute lymphocytic leukemia (ALL), T cell lymphoma (TCL), acute myeloid leukemia (AML), hairy cell leukemia (HCL), Hodgkin's Lymphoma (HL), chronic myeloid leukemia (CML), and nodular lymphocyte-predominant Hodgkin lymphoma.

30. The method of claim 14, wherein the one or more effector and/or functional group is an antibody or a fragment thereof.

31. The method of claim 14, wherein the one or more effector and/or functional groups is a metal chelator, a metal chelator complexed to a metal radioisotope, or a radioactive isotope.

32. The method of claim 14, wherein the one or more effector and/or functional groups is a cytotoxic agent.
